(19) United States
(12) Patent Application Publication
(10) Pub. No.: US 2011/0313258 A1
(43) Pub. Date: Dec. 22, 2011

(54) METHOD AND APPARATUS FOR SOLICITING AN EXPERT OPINION FROM A CARE PROVIDER AND MANAGING HEALTH MANAGEMENT PROTOCOLS

(75) Inventors: Gopal Krishan Chopra, New York, NY (US); Manju Rattan Chopra, New York, NY (US); Alex Veilleux, Jersey City, NJ (US)

(73) Assignee: Dauphin Health, Inc., New York, NY (US)

(21) Appl. No.: 13/161,342

(22) Filed: Jun. 15, 2011

Related U.S. Application Data
(60) Provisional application No. 61/355,533, filed on Jun. 16, 2010.
(60) Provisional application No. 61/391,489, filed on Oct. 8, 2010.

(51) Int. Cl. A61B 5/00

(52) U.S. Cl. 600/300

(57) ABSTRACT

A computer-implemented method of facilitating communication between a care giver and a care provider. The method includes receiving identifications of one or more of primary complaints and selecting data collection questions based at least in part on the identified primary complaint(s). The questions are presented to the care giver in non-expert language and configured to help identify a medical problem associated with the medical issue, but not to diagnosis the medical problem. After responses to the questions are received from the care giver, an Intelligent Resident Note is constructed and displayed to the care provider. The care provider enters or selects a diagnosis and one or more medical protocols for inclusion in a management protocol. The management protocol may include one or more instructions related to treat the medical problem that are sent to the care giver. The care giver confirms when each instruction has been performed.

### CROSS REFERENCE TO RELATED APPLICATION(S)

This application claims the benefit of U.S. Provisional Application No. 61/355,533, filed Jun. 16, 2010, and U.S. Provisional Application No. 61/391,489, filed Oct. 8, 2010, both of which are incorporated herein by reference in their entireties.

### BACKGROUND OF THE INVENTION

1. Field of the Invention

The present invention is directed generally to methods and systems for requesting an expert opinion, receiving the expert opinion in response to the request, and managing implementation of any guidance received with the expert opinion.

2. Description of the Related Art

Parents and consumers of health care find it difficult to communicate medical issues with doctors. Health care providers require additional information that telephonic services cannot provide, including pictures, video, and relevant health data (both current and historic). Doctors (or Experts) are burdened with inefficient data delivery and search systems (in both paper and electronic form). There is a lack of prioritization of work inflow in the practice and inappropriate triaging of clinical issues. Further, healthcare today does not have systems for effectively evaluating or measuring patient compliance to health management protocols, and performance provided by providers of health care. Presently available systems also fail to effectively implement best practices related healthcare.

Therefore, a system is need that facilitates communication between care givers and care providers. A system capable of soliciting information from care givers, including data files and data streams (e.g., image files, video files, audio files, and the like), would be particularly desirable. Searching healthcare related data and generating reports based on data collected for the purposes of delivering care and evaluating care provider performance are also desirable features. Tracking a care giver's compliance to a health management protocol would also be beneficial. The present application provides these and other advantages as will be apparent from the following detailed description and accompanying figures.

### BRIEF DESCRIPTION OF THE SEVERAL VIEWS OF THE DRAWING(S)

FIG. 1 is an illustration of a system configured to facilitate communication between an expert (e.g., a Care Provider) and an entity (e.g., a Care Giver) requesting an expert opinion from the expert.

FIG. 2 is a flow diagram of a method of configuring a non-expert user interface for use by the Care Giver illustrated in FIG. 1.

FIG. 3A-1 is a flow diagram of a first portion of a method of creating a new Opinion Request.

FIG. 3A-2 is a flow diagram of a second portion of the method of creating the new Opinion Request.

FIG. 3B is a flow diagram of a method of constructing a list of preferred care providers that may be used to select a care provider for the Opinion Request created by the method illustrated in FIGS. 3A-1 and 3A-2.

FIG. 3C is an illustration of a non-expert user interface configured to display the list of selectable Primary Complaints to the Care Giver and receive a selection of one or more of the selectable Primary Complaints from the Care Giver.

FIG. 3D is an illustration the non-expert user interface configured to display a question (“When did the fever start?”) to the Care Giver and a graphical display (e.g., a rotatable drum) providing selectable options (e.g., dates/times) that may be used to respond to the question.

FIG. 3E is an illustration the non-expert user interface configured to display a question (“What is his Temperature?”) to the Care Giver and a graphical display (e.g., a rotatable drum) providing selectable options (e.g., temperatures) that may be used to respond to the question.

FIG. 3F is an illustration the non-expert user interface configured to display a plain language description of the medical issue, selectable buttons that when selected initiate an upload of a data file, a selectable button that when selected displays a text box into which text may be entered, and selectable buttons for indicating the Care Giver wishes to select a preferred care provider or a non-preferred care provider.

FIG. 4 is a flow diagram of a method of editing an Opinion Request created by the method illustrated in FIGS. 3A-1 and 3A-2.

FIG. 5 is a flow diagram of a method of assigning the Opinion Request created by the method illustrated in FIGS. 3A-1 and 3A-2 to a different care provider.

FIG. 6A is a flow diagram of a method of configuring the non-expert user interface to display at least a portion of the Expert Opinion to the Care Giver.

FIG. 6B is a flow diagram of a method of tracking compliance or adherence of the Care Giver to one or more Medical Protocols included in a Management Protocol portion of the Expert Opinion.

FIG. 6C is an illustration the non-expert user interface configured to display at least a portion of the Expert Opinion.

FIG. 7A is a flow diagram of a method of requesting a second or specialist opinion for use with the non-expert user interface operated by the Care Giver.

FIG. 7B is a flow diagram of a method of updating a Health Profile associated with a Care Recipient for use with the non-expert user interface operated by the Care Giver.

FIG. 7C is an illustration the non-expert user interface configured to display inputs (illustrated as “thumbs up” and “thumbs down” symbols) for receiving observations related to the Care Recipient.

FIG. 7D is an illustration the non-expert user interface configured to display an exemplary Developmental Report View.

FIG. 7E is an illustration the non-expert user interface configured to display a health journal (e.g., an asthma journal) having a graphical scale input with a plurality of selectable symbols (illustrated as cartoon faces).

FIG. 7F is an illustration the non-expert user interface configured to display a Journal Summary View that includes a graph of the data entered.

FIG. 8 is a flow diagram of a method of constructing an Intelligent Resident Note and notifying the Care Provider of a new Opinion Request (or Live Case).

FIG. 9A is a flow diagram of a method of configuring an expert user interface for use by the Care Provider illustrated in FIG. 1.

FIG. 9B-1 is an illustration the expert user interface configured to display a first portion of an Intelligent Resident Note.

FIG. 9B-2 is an illustration the expert user interface configured to display a second portion of the Intelligent Resident Note and selectable options for processing the Opinion Request for which the Intelligent Resident Note was generated.

FIG. 10A is a flow diagram of a first embodiment of a method of providing a medical protocol in response to an Opinion Request.

FIG. 10B-1 is a flow diagram of a first portion of a second embodiment of a method of providing a medical protocol in response to the Opinion Request.

FIG. 10B-2 is a flow diagram of a second portion of the second embodiment of the method of providing a medical protocol in response to the Opinion Request.

FIG. 10C is an illustration the expert user interface configured to display multiple selectable medical protocols associated with a diagnosis.

FIG. 10D is an illustration the expert user interface configured to display default values and information associated with a selected medical protocol.

FIG. 11 is a flow diagram of a method of requesting additional information from the Care Giver.

FIG. 12 is a flow diagram of a method of recommending or suggesting an investigation (e.g., a laboratory test) to the Care Giver.

FIG. 13 is a flow diagram of a method of recommending or suggesting a second opinion or review by a specialist to the Care Giver.

FIG. 14 is a flow diagram of a method of processing an Opinion Request identified by the Care Provider as urgent.

FIG. 15 is a flow diagram of a method of extracting information from the Expert Opinion for storage by the system and generating a plain language version of the Expert Opinion for display by the non-expert user interface.

FIG. 16 is a diagram of a hardware environment and an operating environment in which the computing devices of the system of FIG. 1 may be implemented.

FIG. 17 is a functional block diagram illustrating a mobile communication device that may be used to implement the mobile computing devices of the system of FIG. 1.

### DETAILED DESCRIPTION OF THE INVENTION

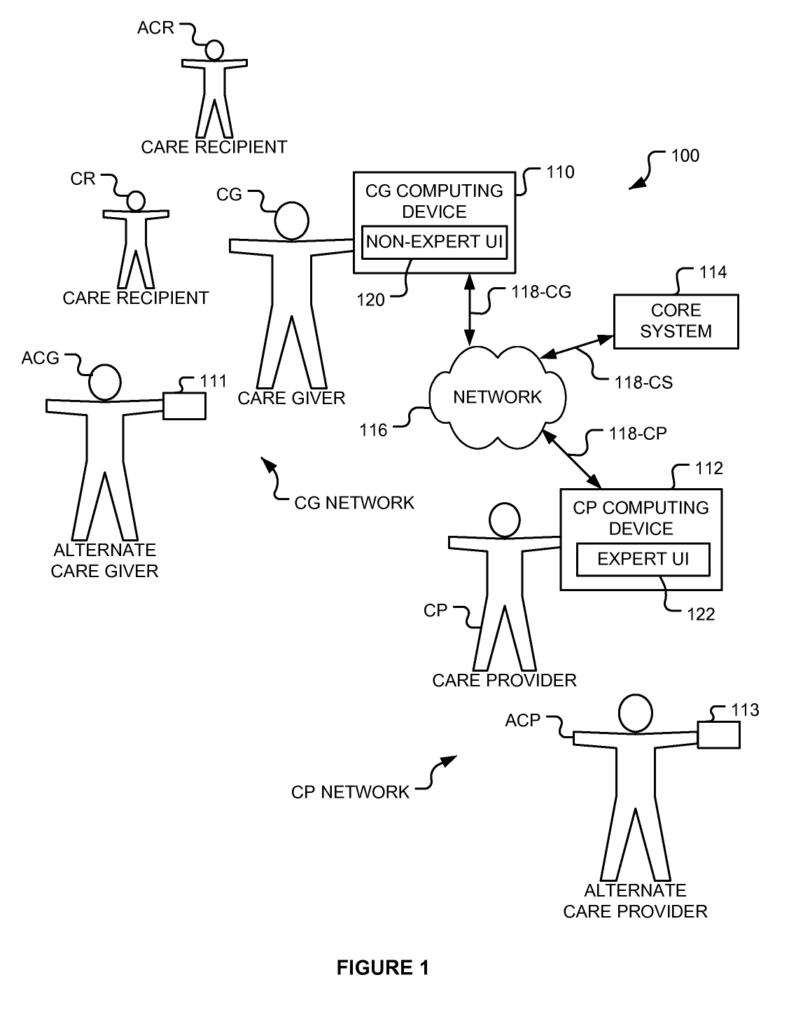

Referring to FIG. 1, the present application describes a system 100 configured to facilitate communication between an expert (e.g., a physician, an architect, an attorney, a veterinarian, and the like) and a person or system requesting an expert opinion from the expert. In some embodiments, the requester of the opinion may seek the expert opinion on behalf of a third party (e.g., a minor child, an employer, a client, a pet, and the like). The system 100 facilitates communication been the requester and the expert at least in part by providing a non-expert user interface 120 to be viewed by the requester and an expert user interface 122 to be viewed by the expert. As will be described below, the system 100 translates communications received from the requester (in lay or plain language) into expert language to be reviewed by the expert via the expert user interface 122. Conversely, the system 100 translates communications received from the expert in expert language into plain language to be reviewed by the requester via the non-expert user interface 120.

### Definition of Terms

Unless defined otherwise, technical terms used herein have the same meaning as commonly understood by one of ordinary skill in the art to which this invention belongs. For purposes of the present invention, the following terms are defined below.

Alternate Care Giver: in a medical implementation, a person or entity, identified by a Care Giver (defined below) as authorized to deliver care to a Care Recipient (defined below), input information associated with the Care Recipient into the system 100, and/or access information associated with the Care Recipient stored in the system 100.

Alternate Care Provider: in a medical implementation, a person or entity, identified by a Care Provider (defined below) as authorized to provide an expert opinion to the Care Giver, input information associated with the Care Recipient into the system 100, and/or access information associated with the Care Recipient stored in the system 100.

Care Giver: in a medical implementation, the requester of an Expert Opinion (defined below). The Care Giver functions as an intermediary who translates and characterizes the issue in the Opinion Request and after receiving the Expert Opinion, may institute the care or management protocol provided by the Care Provider.

Care Provider: in a medical implementation, the expert from whom the Expert Opinion is sought.

Care Recipient: in a medical implementation, a party with whom the Expert Opinion relates. The Expert Opinion may include a Management Protocol (defined below).

Compliance: in a medical implementation, adherence by the Care Giver to a Management Protocol or other guidance received from an Expert.

Conformance: the result of providing a Management Protocol that follows and conforms to known and published expert sources and governing bodies (EBM) or best practice.

Conventional Resident Note: note made by a trained or expert aide, where the aide is able to gather appropriate and necessary details for an expert in the language and format of that expert. In conventional healthcare systems, the Resident Note is delivered to the expert who provides an Expert Opinion based at least in part on the information included in the Resident Note.

eCare: the electronic delivery of health care. Examples of aspects of health care that can be facilitated electronically include but are not limited to the issuance of prescriptions, recommendations for investigations/tests, and recommendation of management protocols.

Enhanced Dialogue: a structured logical Question and Answer (“Q&A”) that delineates an issue (e.g., a medical issue) by collecting data that an expert will use to determine potential causes of the issue (e.g., to arrive at a diagnosis) and deliver guidance (e.g., a Management Protocol) to a Requester of an Expert Opinion such that the Requester can provide information with greater specificity than the Requester would otherwise provide. An Enhanced Dialogue is implemented by the system 100, which depending upon implementation details, may provide information related to prescriptions, investigations, tests, referrals, actions to be performed, and the like to the expert. The Enhanced Dialogue may also implement protocols that help deliver any of the services (e.g., prescription, investigation, and so forth) mentioned above.

Expert Opinion: a response to an Opinion Request (defined below). In a medical implementation, the Expert Opinion may include a Management Protocol formulated by the Care Provider based on the Care Provider's understanding and conclusion with respect to the issue also known as a diagnosis. Herein, the process of receiving the Expert Opinion (e.g., from a care provider) may be referred to as an mConsult.

Expert Language: Specialized language that is easily understood and processed by an expert. Expert language includes jargon, technical terms, terms of art, and terms and phrases known to an expert but not typically widely, or commonly known by non-experts. Expert language may be presented in a format that is familiar to an expert. The intent of expert language is to make information efficient and easy to process by those familiar with it. However, expert language may be difficult, if not impossible, for those unfamiliar with it (e.g., lay persons) to process and/or understand.

Health Album: a collection of health profiles, each storing relevant health data for a Care Recipient collected via the non-expert user interface. This health data may include biometric data, treatment response data, and other health event data or mVisit details.

Intelligent Resident Note: a Resident Note generated automatically by the system 100.

Live Case: an Opinion Request submitted to the system 100 for processing thereby.

Management Protocol: a compilation of Medical Protocols included in an Expert Opinion provided in response to an Opinion Request.

Medical Protocol: treatment management process for a particular Primary Complaint or related issue that may be concurrent with or preventative of an imminent or possible complication. A Medical Protocol may include instructions and guidance connected and validated against known expert sources and governing bodies based on clinical evidence or outcome (known as evidence based medicine in context of best practices in health care).

Opinion Delivery: the act of providing the Expert Opinion via an expert user interface displayed by a computing device (e.g., a mobile device such as a smart telephone).

Opinion Request: a request for an Expert Opinion submitted via a non-expert user interface displayed by a computing device (e.g., a mobile device such as a smart telephone). Herein, the process of receiving the Opinion Request (e.g., from a care giver) may be referred to as an mVisit.

Plain Language or Non-Expert Language: Language that is simple, clear, direct, and uses common words. Plain language is free of jargon and rarely used words and terms, and comes straight to the point being addressed. Plain language may be presented in a format that is familiar to a non-expert. The intent of plain language is to make information accessible, especially to those unfamiliar with expert language (defined above).

Primary Complaint (“PC”): an issue identified in the Opinion Request as the issue for which the Expert Opinion is sought. The PC may be described in either expert language (e.g., in the expert user interface), plain language (e.g., in the non-expert user interface), and/or using symbols. Optionally, the PC may be associated with one or more data files or data streams that store information (such as images files, audio files, video files, and the like).

Stored Case: Tagged data stored by the system 100 and associated with the third party (e.g., in a medical implementation, a Care Recipient) for whom the Expert Opinion was sought. In a medical implementation, the tagged data is stored in a health profile associated with a Care Recipient.

Symptom algorithm: a process of traversing at least a portion of a data structure (e.g., a tree) containing questions selected and organized to define, clarify, and/or better characterize the PC but not to diagnosis a medical issue associated with the PC.

### Overview

The system 100 is configured to facilitate communication between an expert (e.g., a physician, an architect, an attorney, a veterinarian, and the like) and a person requesting an expert opinion from the expert. In some embodiments, the requester of the opinion may seek the expert opinion on behalf of a third party (e.g., a minor child, an employer, a client, a pet, and the like). The figures illustrate an embodiment of the system 100 configured for use in a medical situation. However, through application of ordinary skill in the art to the teachings provided herein, the system 100 may be configured for use in other situations to obtain different types of opinions. For example, the system 100 may be configured to facilitate communication between a parent (acting as a Care Giver), caring for a child (acting as a Care Recipient), and a pediatric doctor (acting as a Care Provider).

FIG. 1 illustrates the system 100 configured for a medical situation in which the system 100 is used to facilitate communication between a Care Provider “CP” (e.g., a physician), and at least one of a Care Giver “CG” (e.g., a parent) and a Care Recipient “CR” (e.g., a patient). As is appreciated by those of ordinary skill in the art, depending upon circumstances, either a Care Recipient “CR,” or a Care Giver “CG” acting on behalf of the Care Recipient, may communicate with the Care Provider “CP.” For the sake of brevity, the present description will describe the system 100 as being operated by the Care Giver “CG.” However, as is appreciated by those of ordinary skill in the art, any of the functions described as being performed by the Care Giver “CG” may alternatively be performed by the Care Recipient “CR” depending upon various circumstances, such as the capabilities (e.g., age, health, mental capacity, physical ability, and the like) of the Care Recipient “CR.”

In particular implementations, the Care Giver “CG” may identify one or more Alternate Care Givers (e.g., an Alternate Care Giver “ACG”) as members of a Care Giver Network “CG NETWORK.” The members of the Care Giver Network “CG NETWORK” are authorized to deliver care to the Care Recipient “CR,” input information associated with the Care Recipient “CR” into the system 100, and/or access information associated with the Care Recipient “CR” stored in the system 100.

In particular implementations, the Care Provider “CP” may identify one or more Alternate Care Providers (e.g., an Alternate Care Provider “ACP”) as members of a Care Provider Network “CP NETWORK.” By way of another non-limiting example, the Care Giver “CG” may identify one or more Alternate Care Providers as being members of the Care Provider Network “CP NETWORK.” The members of the Care Provider Network “CP NETWORK” are authorized to provide expert opinions to the Care Giver “CG,” input information associated with the Care Recipient “CR” into the system 100, and/or access information associated with the Care Recipient “CR” stored in the system 100. Depending upon the implementation details, care providers other than those in the Care Provider Network “CP NETWORK” may also be authorized to perform these functions.

Members of the Care Giver Network “CG NETWORK” may also use the system 100 to seek Expert Opinions on behalf of the Care Recipient “CR,” and/or refer existing requests for Expert Opinions to members of the Care Provider Network “CP NETWORK.” For example, if a licensed health practitioner has been designated as an Alternate Care Giver “ACG,” the licensed health practitioner can use the system 100 to seek an Expert Opinion on behalf of the Care Recipient “CR,” and/or refer an existing request for an Expert Opinion to a peer (e.g., the Alternate Care Provider “ACP”) who is designated in the system 100 as a member of the Care Provider Network “CP NETWORK.”

The Care Giver “CG” may be responsible for providing care to more than one Care Recipient “CR” (e.g., an Alternate Care Recipient “ACR”). For example, a parent may be responsible for providing care to more than one child.

The system 100 includes a centralized core system 114 in communication with a CG computing device 110 operated by the Care Giver “CG” and a CP computing device 112 operated by the Care Provider “CP.” The system 100 provides communication between the Care Giver “CG” (via the CG computing device 110) and the Care Provider “CP” (via the CP computing device 112).

The CG computing device 110 and/or the CP computing device 112 may be implemented as mobile devices, such as cellular telephones, personal data assistants, and the like. Alternatively, the CG computing device 110 and/or the CP computing device 112 may be implemented as stationary computing devices, such as conventional personal computers, and the like.

The core system 114 may be implemented using one or more computing devices, including one or more conventional web servers.

The core system 114 is connected to the CG computing device 110 and the CP computing device 112 by at least one network 116 (e.g., a cellular network, a Local Area Network, a Wide Area Network, the Internet, and the like). The CG computing device 110 is connected to the network 116 by a communication link 118-CG, the CP computing device 112 is connected to the network 116 by a communication link 118-CP, and core system 114 is connected to the network 116 by a communication link 118-CS. The communication links 118-CG, 118-CP, and 118-CS may each be implemented as a mobile connection, a wireless connection, a wired connection, or a combination of two or more of these types of connections.

The core system 114 generates a non-expert user interface 120 that is displayed to the Care Giver “CG” by the CG computing device 110 and an expert user interface 122 that is displayed to the Care Provider “CP” by the CP computing device 112. Using the non-expert and expert user interfaces 120 and 122, the system 100 implements an Enhanced Dialogue (described in greater detail below) between the Care Giver “CG” and the Care Provider “CP.”

Each of the Alternate Care Givers in the Care Giver Network “CG NETWORK” (e.g., the Alternate Care Giver “ACG”) may operate a computing device 111 configured to communicate with the core system 114. The computing device 111 may be implemented using any computing device suitable for implementing the CG computing device 110. The computing device 111 is operable to display the non-expert user interface 120 generated by the core system 114. However, the non-expert user interface 120 displayed to the Alternate Care Giver “ACG” may perform only a subset of functions available to the Care Giver “CG.” The non-expert user interface 120 may allow the Care Giver “CG” to set access rights for the Alternate Care Giver “ACG.” For example, a parent (acting as the Care Giver “CG”) may grant access rights to a nanny (acting as the Alternate Care Giver “ACG”) to view Opinion Requests created by the Care Giver “CG.” However, the parent may restrict the nanny's access such that the nanny cannot create new Opinion Requests.

Each of the Alternate Care Providers in the Care Provider Network “CP NETWORK” (e.g., the Alternate Care Provider “ACP”) may operate a computing device 113 configured to communicate with the core system 114. The computing device 113 may be implemented using any computing device suitable for implementing the CP computing device 112. The computing device 113 is operable to display the expert user interface 122 generated by the core system 114. However, the expert user interface 122 displayed to the Alternate Care Provider “ACP” may perform only a subset of functions available to the Care Provider “CP.” The non-expert user interface 120 may allow the Care Provider “CP” to set access rights for the Alternate Care Provider “ACP.”

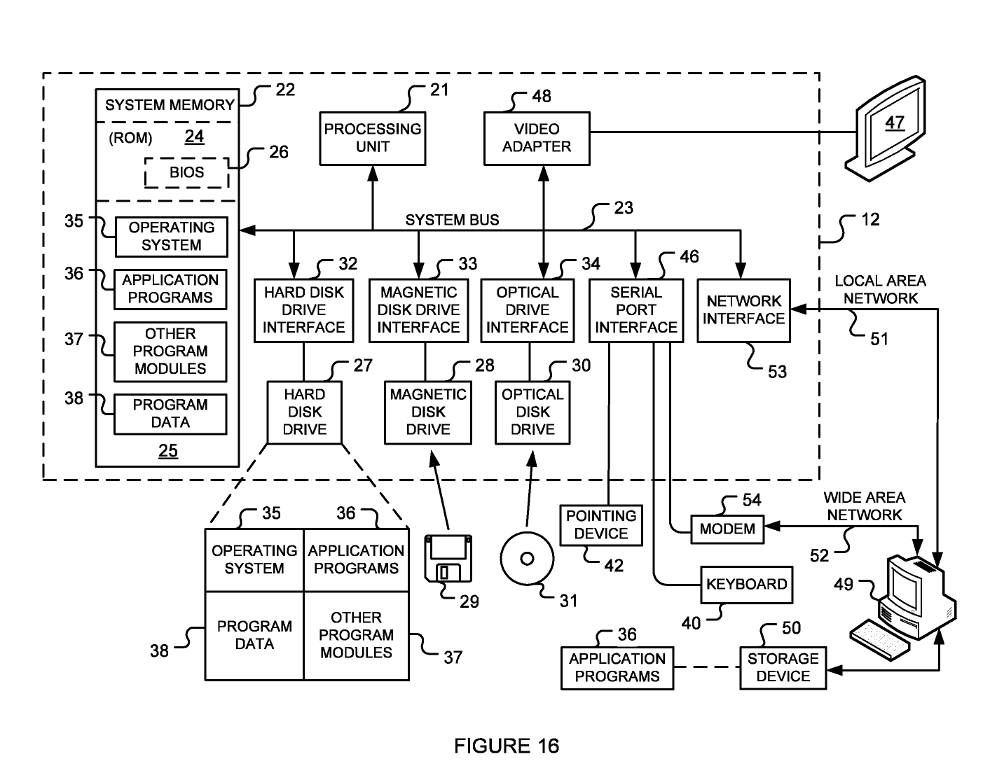

FIG. 16 (described below) illustrates an example of a suitable computing environment for implementing the core system 114, the CG computing device 110, the computing device 111, the CP computing device 112, and/or the computing device 113.

As explained above, the Enhanced Dialogue (described in greater detail below) may be conducted between (1.) the Care Giver “CG” and the Care Provider “CP,” (2.) the Alternate Care Giver “ACG” and the Care Provider “CP,” (3.) the Care Giver “CG” and the Alternate Care Provider “ACP,” and/or (3.) the Alternate Care Giver “ACG” and the Alternate Care Provider “ACP.”

Generally, the Care Giver “CG” will wish to contact the Care Provider “CP” to seek help with an issue (such as an illness, symptom, or question). The system 100 is configured to receive a request from the Care Giver “CG” via the non-expert user interface 120 displayed on the CG computing device 110. The request is submitted by the CG computing device 110 to the core system 114. In response to this request, the core system 114 is configured to collect information from the Care Giver “CG” (via the non-expert user interface 120) and optionally supplement that information. The non-expert user interface 120 may display graphical elements, such as scales, that are easy for the Care Giver “CG” to comprehend and use to enter data about the Care Recipient “CR.” The core system 114 presents the request and information collected to the Care Provider “CP” via the expert user interface 122 (displayed by the CP computing device 112) for the purposes of soliciting an Expert Opinion from the Care Provider “CP.” The Care Provider “CP” reviews the information displayed and submits the Expert Opinion to the core system 114. The core system 114 forwards the Expert Opinion to the Care Giver “CG” via the non-expert user interface 120 displayed by the CG computing device 110.

The methods described below and illustrated in the figures implement an exemplary Enhanced Dialog between the Care Giver “CG” and the Care Provider “CP” provided by the system 100.

### Method 200

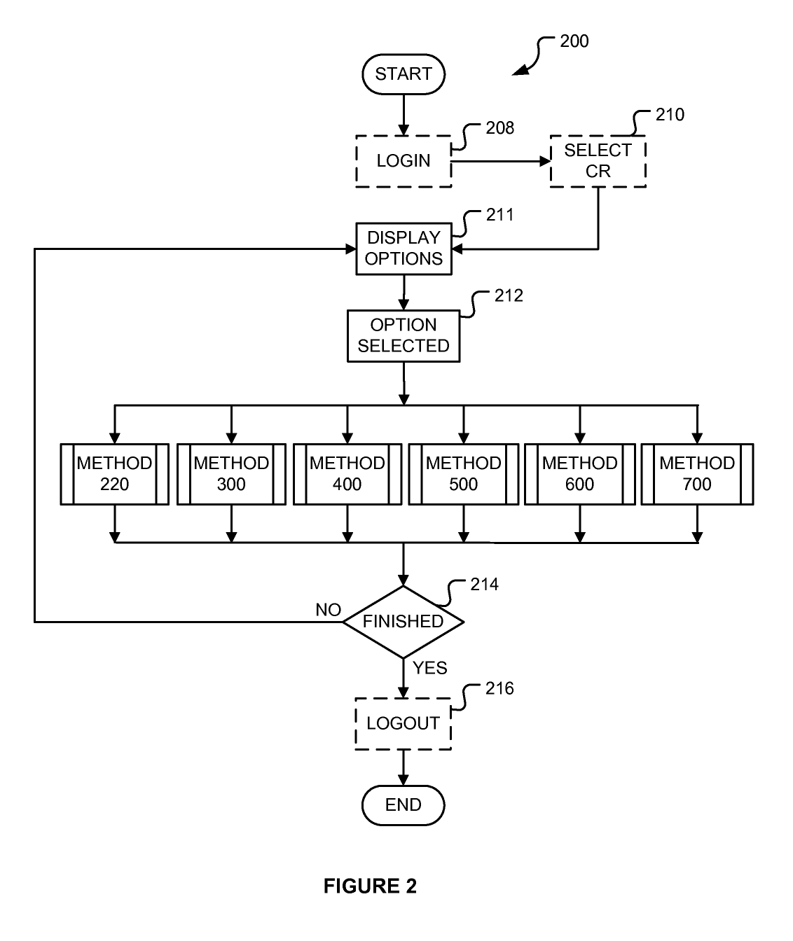

FIG. 2 illustrates a method 200 performed by the system 100 (illustrated in FIG. 1). The method 200 configures the non-expert user interface 120 for display on the CG computing device 110 and receives input from the Care Giver “CG.” Before the method 200 is performed, the Care Giver “CG” may have identified an issue (such as an illness, symptom, or question) about which the Care Giver “CG” wishes to receive an Expert Opinion.

In optional first block 208, the core system 114 receives valid login information from the Care Giver “CG” and in response, displays the non-expert user interface 120 to the Care Giver “CG.” The present disclosure is not limited to use with any particular method of logging into the system 100. After the Care Giver “CG” has successfully logged into the core system 114, the non-expert user interface 120 may display a home page or greeting screen.

In optional block 210, if the Care Giver “CG” is authorized to enter and/or view information related to more than one care recipient, in block 210, the Care Giver “CG” identifies one of the care recipients as the one for which the Opinion Request has been initiated. Thus, in block 210, the system 100 may receive an identification of one of the care recipients for which the Care Giver “CG” is authorized to enter and/or view information from the non-expert user interface 120. For ease of illustration, in the following description of the methods 200, and 220, in block 210, the Care Giver “CG” has identified the Care Recipient “CR.” The non-expert user interface 120 may be configured to display a list of selectable identifiers of care recipients for which the Care Giver “CG” is authorized to enter and/or view information.

In block 211, the non-expert user interface 120 displays at least the following options to the Care Giver “CG:”
1. Create a new Opinion Request;
2. Revise a Live Case (generated from a previously created Opinion Request);
3. Re-Route an Opinion Request to a different Care Provider;
4. Review an Expert Opinion;
5. Request a Second or Specialist Opinion; and
6. Edit Health Profile.

In block 212, the system 100 receives a selection from the Care Giver “CG” of one of the above options via the non-expert user interface 120.

If the Care Giver “CG” selected “Create a new Opinion Request,” a method 220 illustrated in FIGS. 3A-1 and 3A-2 is performed. Then, the system 100 advances to decision block 214.

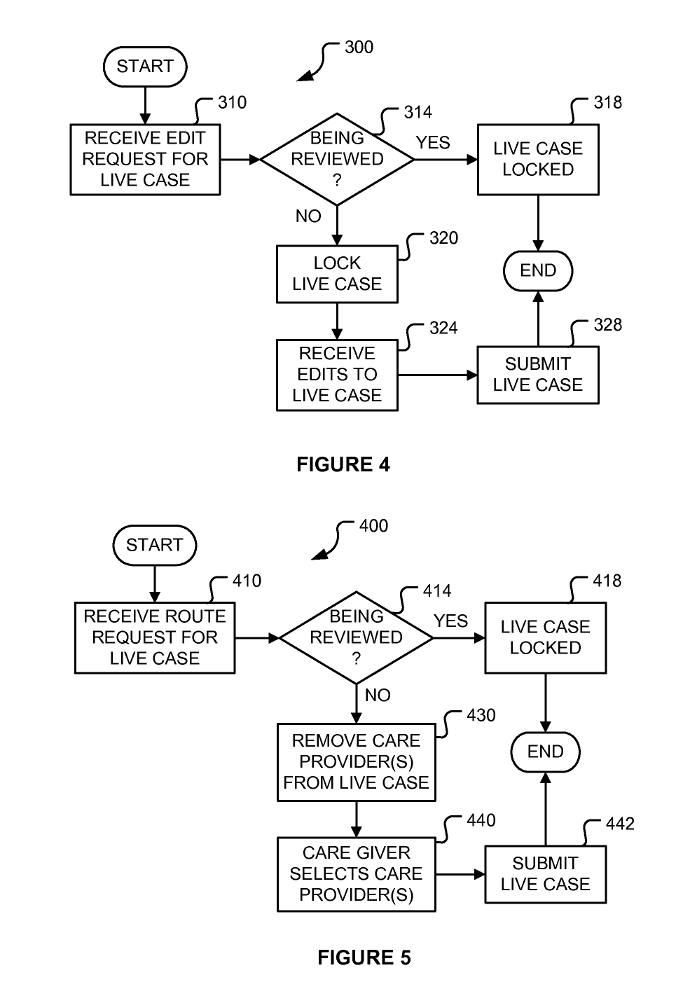

If the Care Giver “CG” selected “Revise a Live Case,” a method 300 illustrated in FIG. 4 is performed. Then, the system 100 advances to decision block 214.

If the Care Giver “CG” selected “Re-Route an Opinion Request to a different Care Provider,” a method 400 illustrated in FIG. 5 is performed. Then, the system 100 advances to decision block 214.

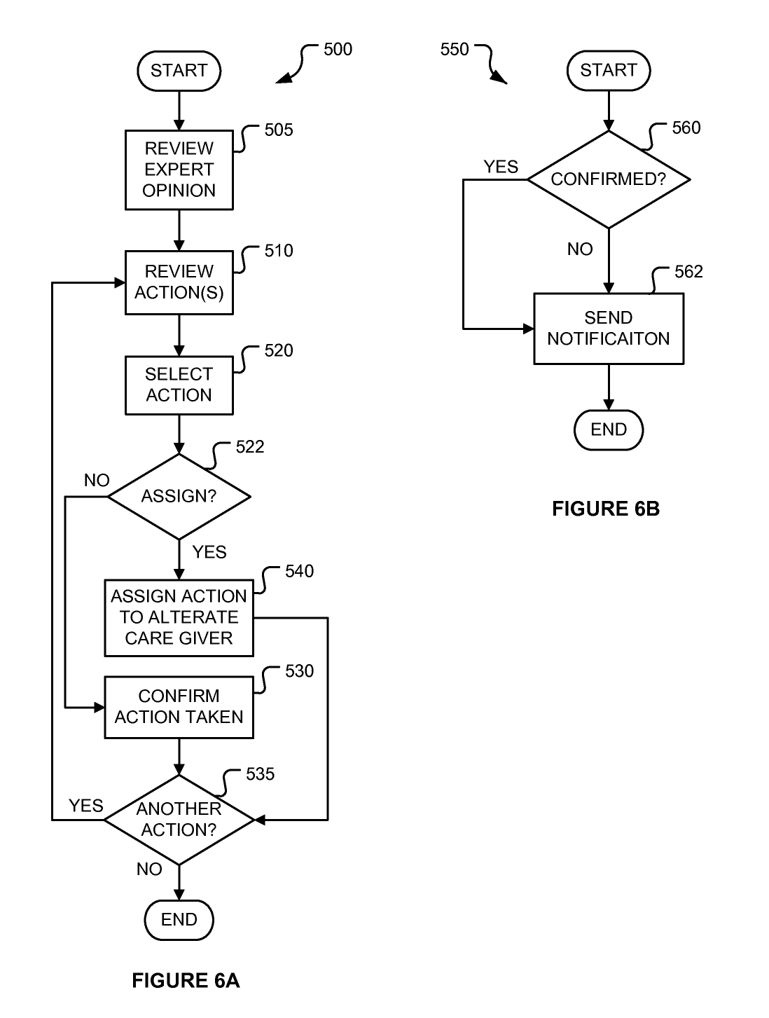

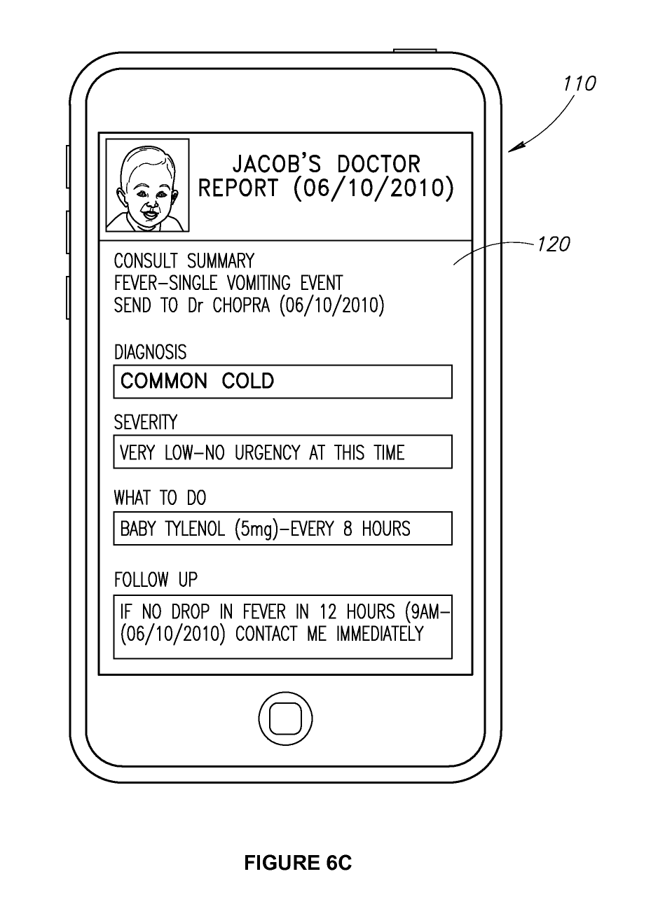

If the Care Giver “CG” selected “Review an Expert Opinion,” a method 500 illustrated in FIG. 6A is performed. Then, the system 100 advances to decision block 214.

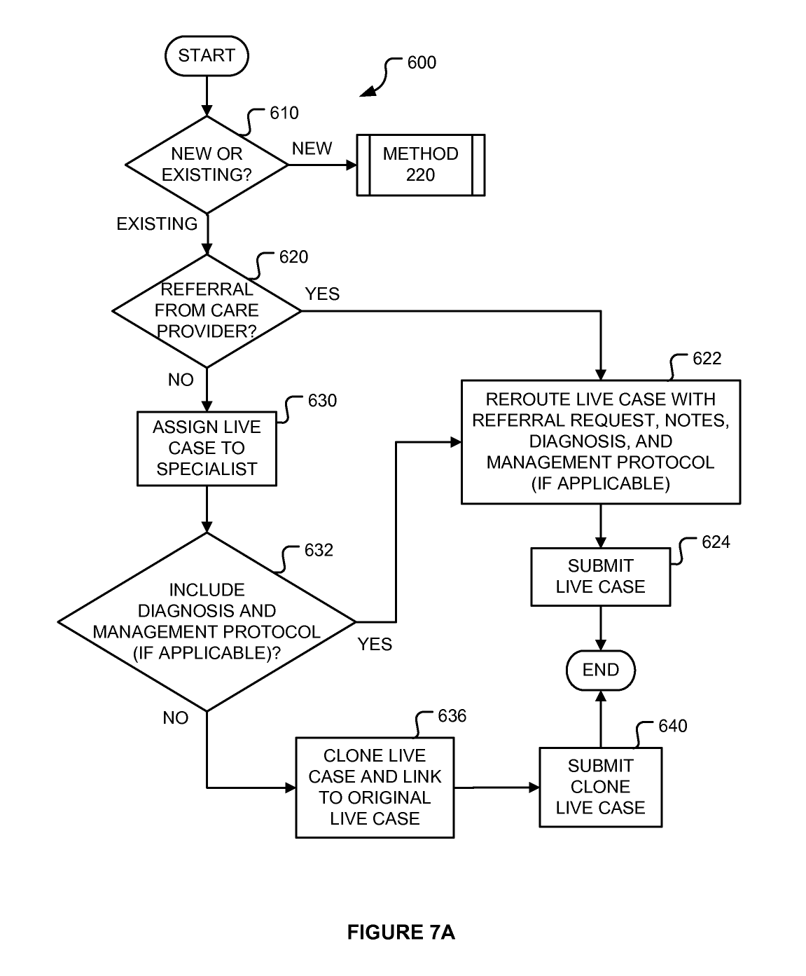

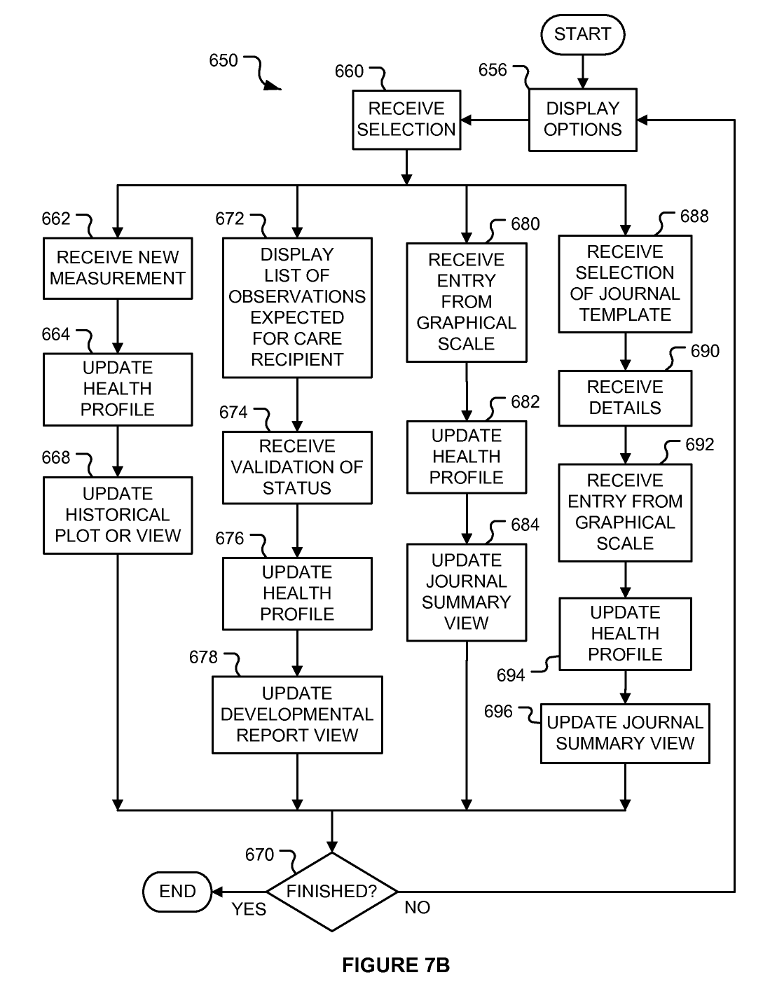

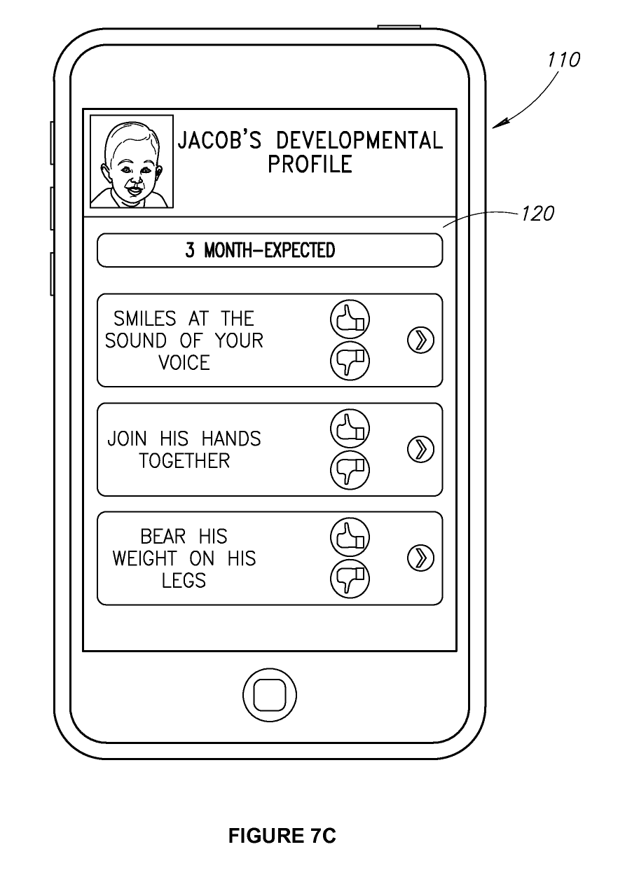

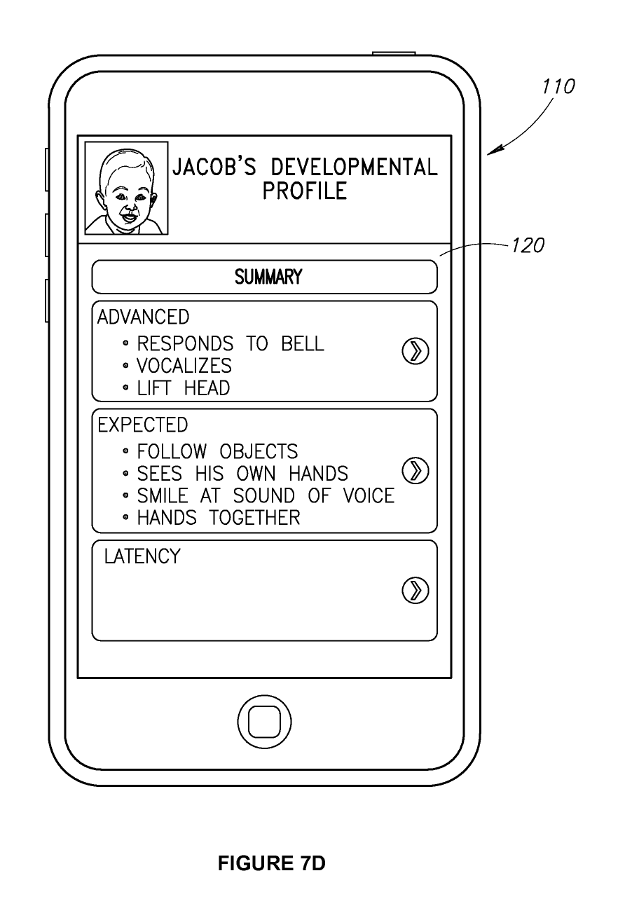

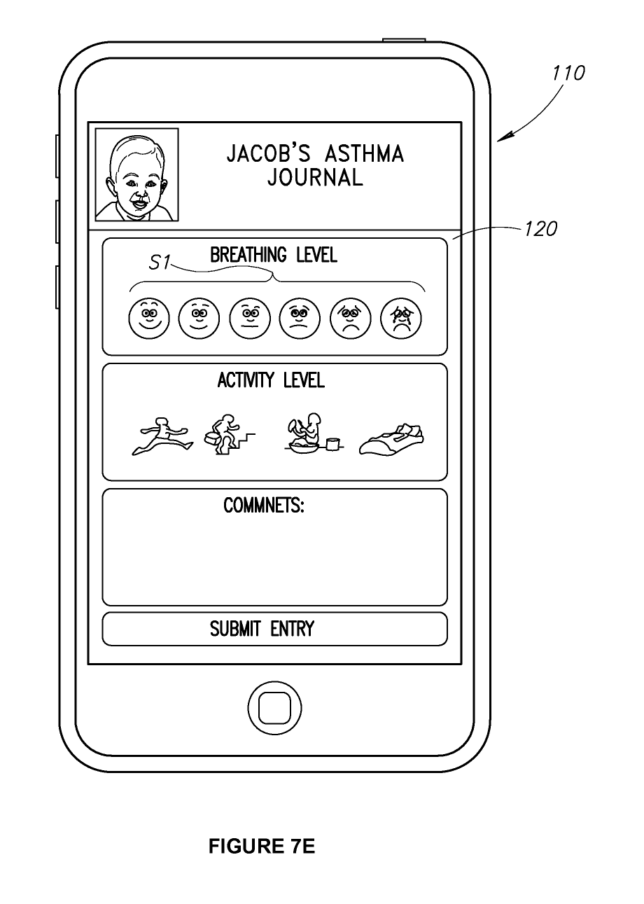

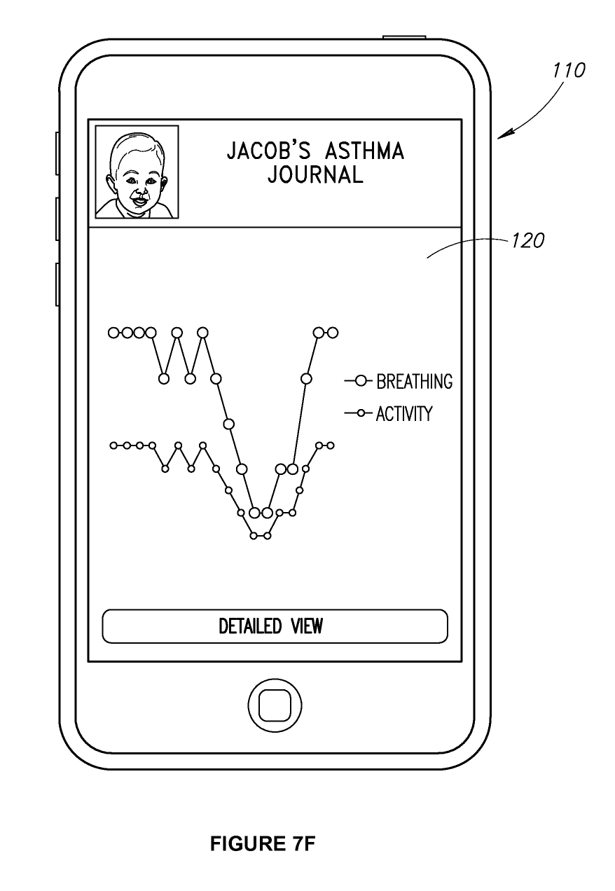

If the Care Giver “CG” selected “Request a Second or Specialist Opinion,” a method 600 illustrated in FIG. 7A is performed. Then, the system 100 advances to decision block 214.

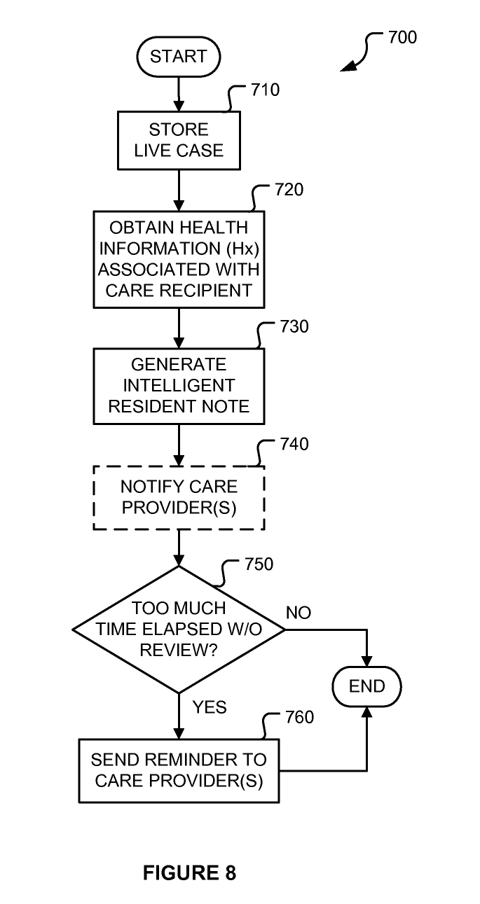

If the Care Giver “CG” selected “Edit Health Profile,” a method 700 illustrated in FIG. 8 is performed. Then, the system 100 advances to decision block 214.

The decision in decision block 214 is “YES” when the Care Giver “CG” has finished selecting options displayed by the non-expert user interface 120. When the decision in decision block 214 is “YES,” the Care Giver “CG” may logout in optional block 216 and the method 200 terminates. The decision in decision block 214 is “YES” when the Care Giver “CG” has not finished selecting options displayed by the non-expert user interface 120. When the decision in decision block 214 is “NO,” the method 200 returns to block 211 whereat the options are displayed to the Care Giver “CG” for selection thereby.

### Method 220: First Embodiment: Create a New Opinion Request

FIGS. 3A-1 and 3A-2 provide a flow diagram of the method 220, which is a first embodiment of a method of creating a new Opinion Request. The method 220 configures the non-expert user interface 120 for display on the CG computing device 110 and receives input from the Care Giver “CG.” In first block 221, a new Opinion Request is initiated.

Then, in optional block 222, the system 100 instructs the non-expert user interface 120 to display a list of preferred care providers and a status associated with each preferred care provider. Exemplary statuses may include “currently online,” “currently offline,” and “will next be online at ______.”

In decision block 223, the system 100 receives an indication from the Care Giver “CG” as to whether the Care Giver would like to update the list of preferred care providers. The decision in decision block 223 is “YES” when the system 100 receives an indication from the Care Giver “CG” that the Care Giver would like to update the list of preferred care providers. On the other hand, the decision in decision block 223 is “NO” when the system 100 receives an indication from the Care Giver “CG” that the Care Giver would not like to update the list of preferred care providers.

When the decision in decision block 223 is “YES,” the method 330 is performed in block 224. As will be explained below, during the performance of the method 330, the Care Giver “CG” selects a care provider.

Because the Opinion Request may be canceled during the performance of the method 330, in decision block 226, the system 100 determines whether the Opinion Request has been canceled. If the Opinion Request has been canceled, the method 220 terminates. On the other hand, if the Opinion Request has not been canceled, the system 100 advances to block 225.

When the decision in decision block 223 is “NO,” the system 100 advances to block 225.

In block 225, the system 100 accesses a health profile associated with the Care Recipient “CR” for the purpose of identifying any chronic conditions the Care Recipient “CR” may have. The health profile includes health information about the Care Recipient “CR,” such as age, gender, and past history. The past history may include Opinion Requests submitted previously for the Care Recipient “CR,” and/or Expert Opinions received previously for the Care Recipient “CR.” Thus, the information in the Health Profile may include information entered manually (e.g., during a registration process) or extracted from previously received Expert Opinions associated with the Care Recipient “CR.” The health profile may be stored in a Health Album.

In block 227, the system 100 formulates a list of selectable Primary Complaints. In block 227, the system 100 may determine whether based on the health profile associated with the Care Recipient “CR,” the Care Recipient “CR” has a chronic condition. If the system 100 determines the Care Recipient “CR” has at least one chronic condition, the system 100 formulates the list of selectable Primary Complaints based at least in part on any chronic conditions identified in the health profile associated with the Care Recipient “CR” and the health profile associated with the Care Recipient “CR.” For example, the list of selectable Primary Complaints may be formulated based at least in part on a combination of chronic conditions, age, demographic details, and other tagged health profile information associated with the Care Recipient “CR.” To assist the Care Giver “CG,” the list may be ordered such that chronic conditions are listed first. If the system 100 determines the Care Recipient “CR” does not have at least one chronic condition, the system 100 formulates a list of selectable Primary Complaints based on the health profile associated with the Care Recipient “CR.” Then, the system 100 advances to block 228.

The list of Primary Complaints is a list of common and probable symptoms, signs and locations of disorders one or more of which may characterize the primary reason for initiating the Opinion Request. In other words, one or more of the selectable Primary Complaints may describe the issue in a broad sense.

The list may be personalized to the Care Recipient “CR.” For example, whether or not any relevant chronic or acute conditions were identified in block 227, the list may be prioritized such that Primary Complaints more likely to be selected appear first. The priority may be based on past events, and/or past history. For example, conditions identified in more recent events may be included in the list and given high priority than conditions identified in earlier occurring events. By way of another example, the priority may be based on demographic information such as age, gender, as well as geolocation. Further, priority may be based on a combination of any of the aforementioned pieces of information.

In block 228, the system 100 instructs the non-expert user interface 120 to display the list of selectable Primary Complaints to the Care Giver “CG.”

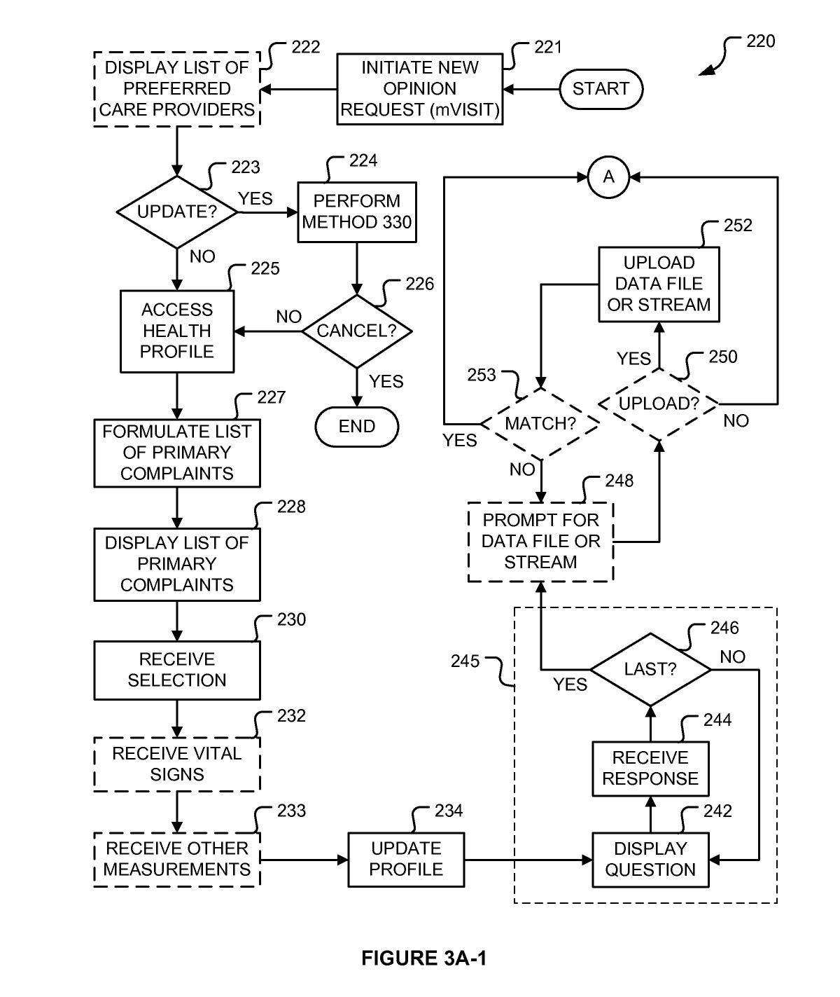

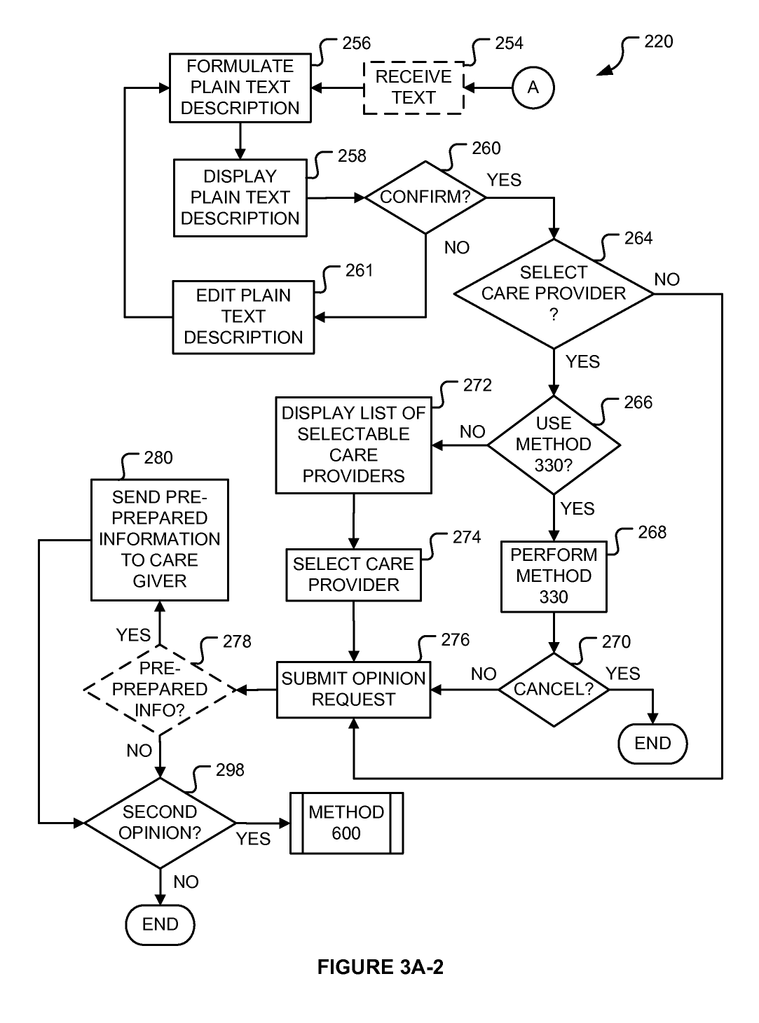

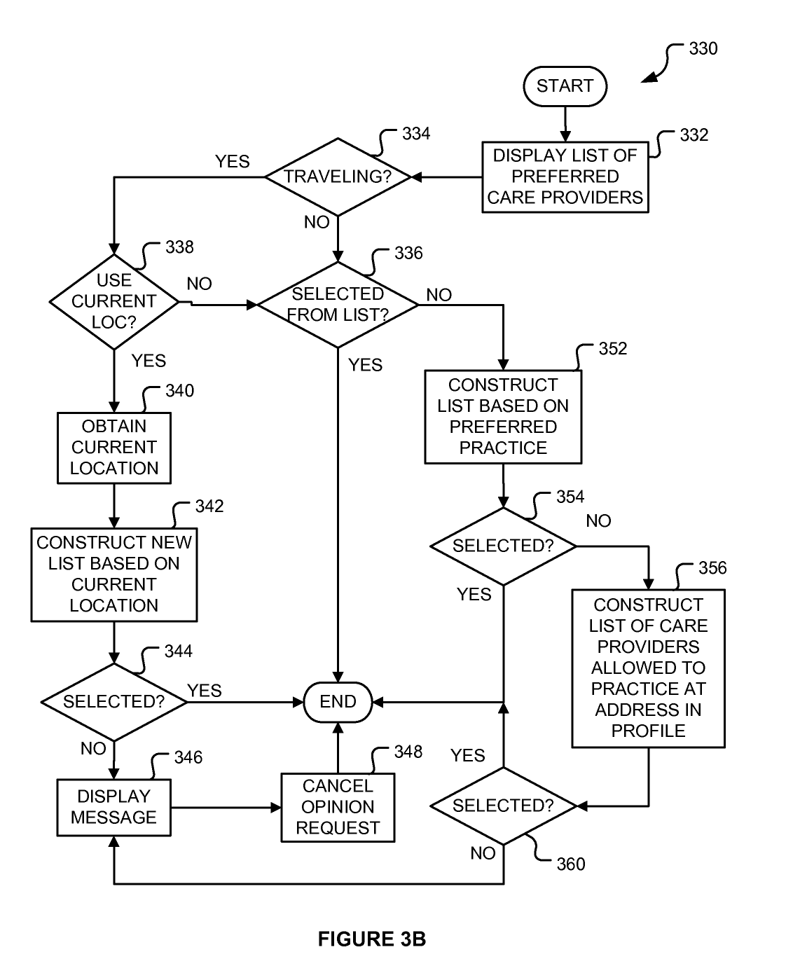

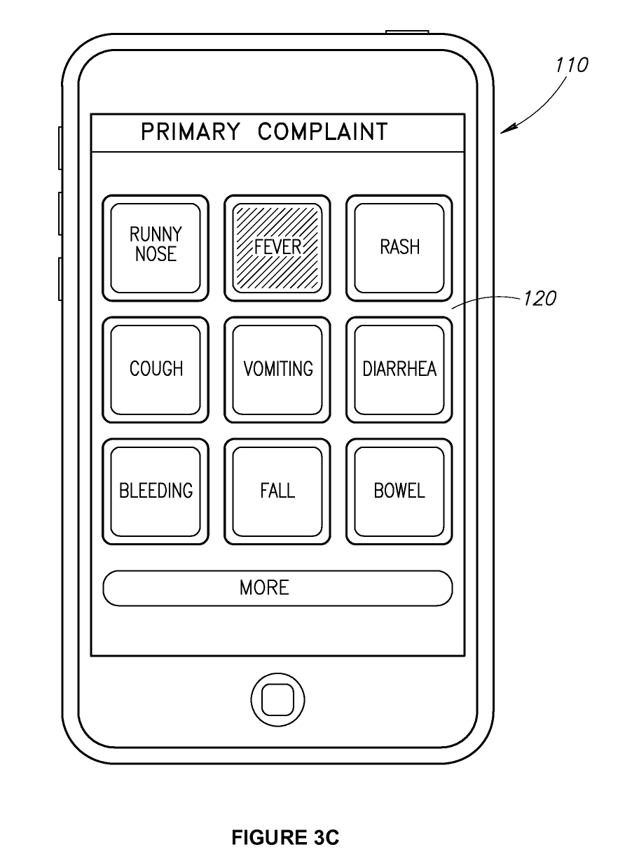

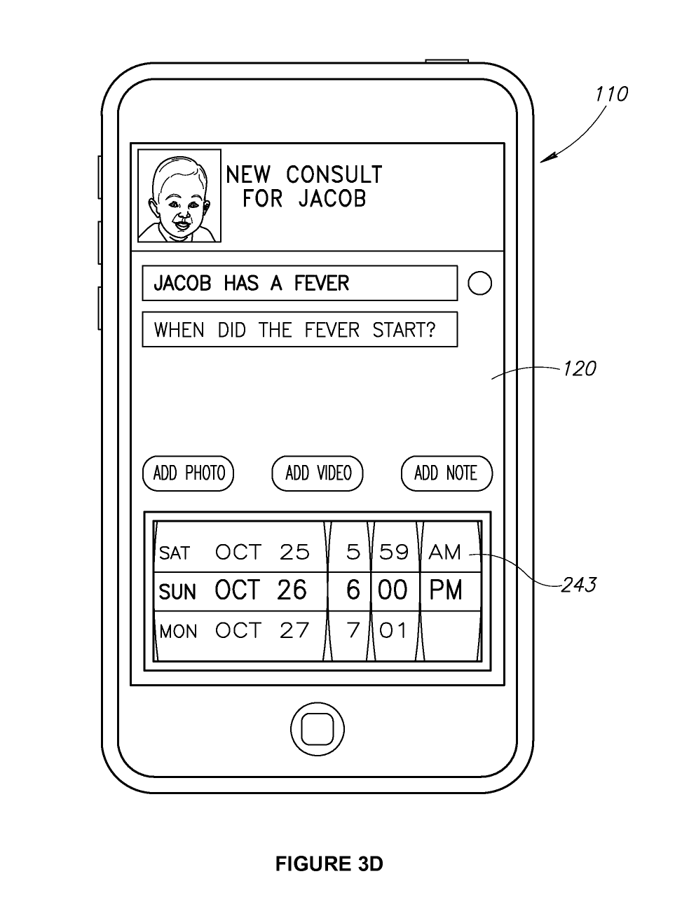

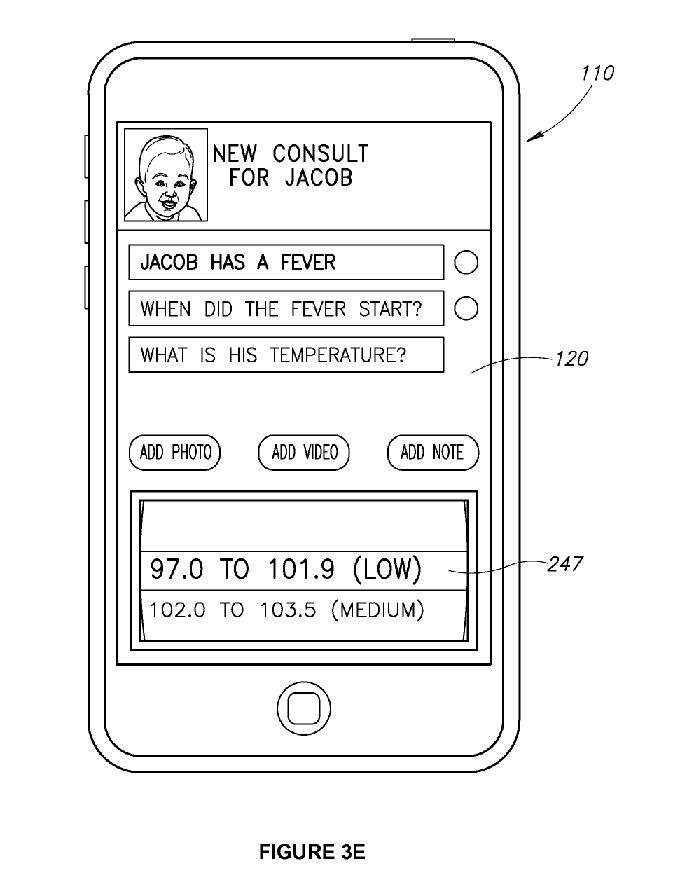

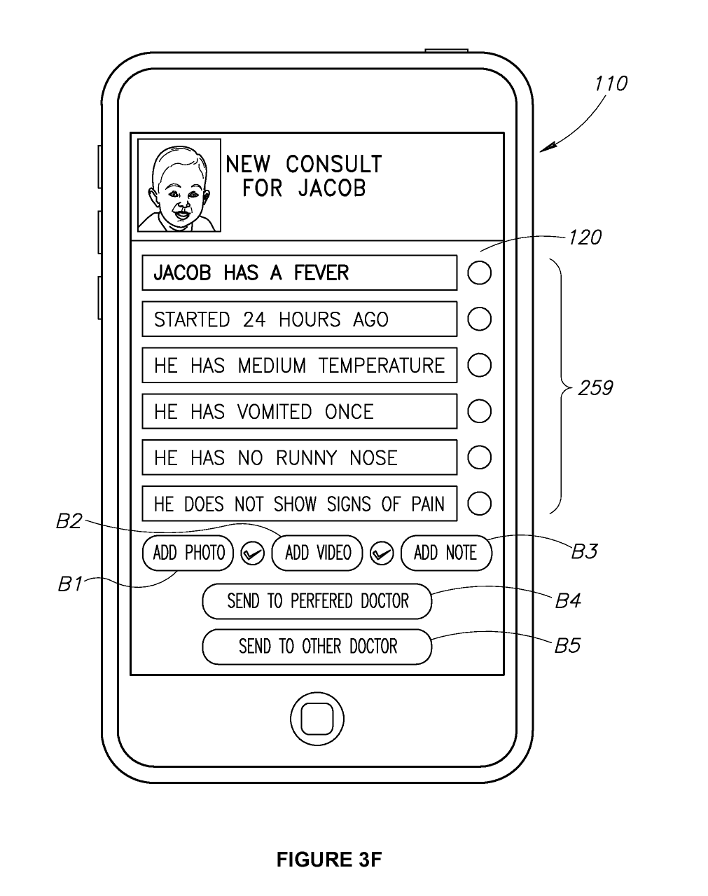

In next block 230, the system 100 receives a selection of one or more of the selectable Primary Complaints from the list. FIG. 3C provides an example of the non-expert user interface 120 configured to display the list of selectable Primary Complaints to the Care Giver “CG.” In FIG. 3C, the non-expert user interface 120 is illustrated receiving a selection from the Care Giver “CG” of the Primary Complaint labeled “FEVER.”

Returning to FIG. 3A-1, in optional block 232, the system 100 receives vital sign and/or other observational information from the care giver computing device 110 that was input into the non-expert user interface 120 by the Care Giver “CG.” Before the vital signs are received, the system 100 may instruct the non-expert user interface 120 to display a request to the Care Giver “CG” requesting that the Care Giver “CG” input the vital sign and/or other observational information for the Care Recipient “CR.” In

Optionally, in block 233, the system 100 receives other data (which may include Vital Sign information like Respiratory Rate, Temperature, O₂Saturation) input into the non-expert user interface 120 by the Care Giver “CG” and/or external medical devices (such as pulse oximeters, digital thermometers, etc.). Such external medical devices may or may not communicate with the CG computing device 100 via wireless communication protocols.). For example, in block 233, data may be input manually or automatically and loaded in the Opinion Request as appropriate with recognition/acknowledgement of the individual (e.g., the Care Recipient “CR”) for which the data is relevant.

In optional block 234, the system 100 determines whether health information (e.g., height, weight, etc.) stored in the health profile associated with the Care Recipient “CR” is current. The system 100 may identity any health information older than a predetermined threshold age as being “out of date.” In particular embodiments, only health information related to physical measurements (such as those affecting mediation dosages) is evaluated. Exemplary physical measurements include height and weight. If any of the health information evaluated is identified as being “out of date.” the system 100 instructs the non-expert user interface 120 to display a request to the Care Giver “CG” requesting that the Care Giver “CG” update the health information determined not to be current (or out of date). When the system 100 receives updated health information (input into the non-expert user interface 120 by the Care Giver “CG”), the system 100 updates the health profiles associated with the Care Recipient “CR” with the updated health information. Then, the system 100 advances to block 242.

In block 242, the system 100 displays one or more questions to the Care Giver “CG.” The system 100 may select the questions to display based at least in part on the Primary Complaint(s) selected. FIG. 3D provides an example of the non-expert user interface 120 configured to display a first question to the Care Giver “CG” (e.g., “When did the fever start?”) and a graphical display 243 (e.g., a rotatable drum) providing selectable options (e.g., dates/times).

Returning to FIG. 3A-1, in block 244, the system 100 receives responses from the Care Giver “CG” to the question(s) presented in block 242.

In decision block 246, the system 100 determines whether it has asked the last question. The decision in decision block 246 is “YES” when the system 100 has asked the last question. On the other hand, the decision in decision block 246 is “NO” when the system 100 has not asked the last question.

When the decision in decision block 246 is “No,” the system 100 returns to block 242 and displays one or more questions. FIG. 3E provides an example of the non-expert user interface 120 configured to display a second question (“What is his temperature?”) to the Care Giver “CG” and a graphical display 247 (e.g., a rotatable drum) providing selectable options (e.g., temperatures).

Returning to FIG. 3A-1, the blocks 242, 244, and 246 may be characterized as implementing a tailored health interrogation process identified by dashed rectangle 245. The tailored health interrogation process 245 may present questions to the Care Giver “CG” in accordance with a predetermined set of questions stored in a tree (described in more detail below). The questions presented may be organized in a tree structure (e.g., using a decision tree algorithm). Thus, subsequent questions presented to the Care Giver “CG” may be based on answers given to previously presented questions. The process of presenting these questions and receiving answers thereto is referred to as an “interrogation of the Care Giver.” Thus, in block 244, the Care Giver “CG” provides information related to the issue.

The questions may include general questions that the physician would ask as well as specific questions drawn from the tree. When traversing the tree, the next question asked may be selected based on the response received to previously asked question. The questions asked may also be selected based at least in part on relevant information in medical history, which includes past events (e.g., records from previous Opinion Requests) and relevant information stored in a health record associated with the Care Recipient “CR.” The questions asked may also be selected based at least in part on pharmaceutical event history associated with the Care Recipient “CR.” There may be branched or concomitant trees for separate processes, for example separate tree for pharmaceutical use and response questions.

The non-expert user interface 120 assists in the collection information from the Care Giver “CG” by asking questions designed to solicit information from the Care Giver “CG” that will help the Care Provider “CP” arrive at an Expert Opinion. For example, the questions may help identify and classify the issue. In other words, the questions presented to the Care Provider “CP” may help frame the issue. Such questions may help not only determine diagnose a medical problem but may also help determine its severity.

The system 100 may be configured to perform automated symbol recognition and associate a symbol with a symptom (such as smiley face for pain). During the interrogation of the Care Giver, the non-expert user interface 120 may display scales where the severity or acuity of an issue is graded (e.g., by color, severity of pain, etc.). The non-expert user interface 120 may receive text, or display a list of selectable options.

When the decision in decision block 246 is “YES,” in optional block 248, the non-expert user interface 120 prompts the Care Giver “CG” for a data file and/or data stream (e.g., storing or encoding a photograph, a video, a sound recording, a text file, and the like). Such data files and/or data streams may be labeled or referred to as “rich media” in the various figures. FIG. 3F illustrates the non-expert user interface 120 configured to display selectable buttons B-1 (for uploading an image file), and B-2 (for uploading a video file) that when selected initiate an upload of a data file.

In the method 220, the block 232, the block 233, the block 234, and the process 245 may be performed in any order and the method is not limited to the order illustrated in FIG. 3A-1.

In optional decision block 250, the system 100 receives an indication from the Care Giver “CG” indicating whether the Care Giver “CG” wishes to upload a data file. The decision in decision block 250 is “YES” when the Care Giver “CG” wishes to upload a data file. On the other hand, the decision in decision block 250 is “NO” when the Care Giver “CG” does not wish to upload a data file.

When the decision in decision block 250 is “YES,” in block 252, the system 100 uploads a data file and/or data stream identified by the Care Giver “CG” and receives information entered by the Care Giver “CG” describing the uploaded data. Thus, in block 252, the system 100 may instruct the non-expert user interface 120 to display a request for information describing the uploaded data and at least one data input configured to receive the information.

In optional decision block 253, the system 100 determines whether the uploaded data file and/or data stream matches the one for which a prompt was displayed in block 248. For example, if the system prompted the Care Giver “CG” for a photograph of the left leg of the Care Recipient “CR” in block 248 and the Care Giver “CG” uploaded a digital photograph of the right leg of the Care Recipient “CR,” the uploaded data file would not match the one for which a prompt was displayed in block 248. In block 253, the system 100 may determine a mismatch has occurred when the information describing the uploaded data does not describe the data file and/or data stream for which a prompt was displayed in block 248.

The decision in decision block 253 is “YES” when the uploaded data file and/or data stream matches the one for which a prompt was displayed in block 248. Otherwise, the decision in decision block 253 is “NO” when the uploaded data file and/or data stream does not match the one for which a prompt was displayed in block 248.

When the decision in decision block 253 is “YES,” the system 100 advances to block 254 (see FIG. 3A-2). When the decision in decision block 253 is “NO,” the system 100 returns to block 248 and prompts the Care Giver “CG” for an additional data file and/or data stream.

The uploaded data file and/or data stream is tagged by the system 100 with the Primary Complaint(s) selected above and other information (e.g., responses received during the tailored health interrogation process 245). When the Expert Opinion has been received for the Opinion Request, the data file is tagged with the Expert Opinion. When the decision in decision block 250 is “NO,” the system 100 advances to block 254 (see FIG. 3A-2).

Turning to FIG. 3A-2, in optional block 254, the system 100 instructs the non-expert user interface 120 to request free-form text from the Care Giver “CG.” For example, in block 254, the non-expert user interface 120 may display a text box into which the Care Giver “CG” may type information (e.g., information that describes the issue or any concerns the Care Giver “CG” may have). FIG. 3F illustrates the non-expert user interface 120 configured to display a selectable button B-3 (for uploading a free-form text note) that when selected display a text box into which the Care Giver “CG” may type information. Returning to FIG. 3A-2, after the Care Giver “CG” enters free-form text or indicates the Care Giver “CG” does not wish to enter such text, in block 256, the system 100 formulates a lay or plain-language description of the issue.

The non-expert user interface 120 may display one or more education links to additional information. If the Care Giver “CG” wants further information, the Care Giver “CG” may select one of these links. For example, the non-expert user interface 120 may display an educational link associated with a question, word, or process. The non-expert user interface 120 may also display a description of what information is being requested or what needs to be done to get the requested information. The education links can be provided in text or multimedia format. For example, the text “pulse rate” may include an education link to a video showing how to measure pulse rate. The education links and/or the information displayed when an education link is selected may be specific to the personalized need of the Care Giver “CG” and/or Care Recipient “CR.” For example, the text “pulse rate” may include an education link to an educational video showing how to measure pulse rate that is specific to the age of the Care Recipient “CR” (e.g., a child).

Further, whenever default values and/or information is displayed by the non-expert user interface 120 and/or the expert user interface 122, the default values and/or information may be are specific to the Care Giver “CG” and/or Care Recipient “CR.” For example, normal parameters for a Care Recipient “CR” may vary based on age. The non-expert user interface 120 and/or the expert user interface 122 may be configured to display normal parameters for the Care Recipient “CR” identified by the Opinion Request or for whom health profile data is being entered.

In block 258, the plain-language description of the issue is presented to the Care Giver “CG” for acceptance. FIG. 3F illustrates the non-expert user interface 120 configured to display the plain-language description 259 of the issue.

Returning to FIG. 3A-2, in decision block 260, the system 100 receives an indication as to whether the Care Giver “CG” has accepted (or confirmed) the plain-language description. The decision in decision block 260 is “YES” when the Care Giver “CG” has confirmed the plain-language description. Otherwise, the decision in decision block 260 is “NO” when the Care Giver “CG” has not confirmed the plain-language description.

When the decision in decision block 260 is “NO,” in block 261, the non-expert user interface 120 is configured to allow the Care Giver “CG” to edit information entered previously into the non-expert user interface 120. Then, the system 100 returns to block 256 to reformulate the plain-language description.

When the decision in decision block 260 is “YES,” in decision block 264, the system 100 determines whether the Care Giver “CG” needs to select a care provider. The decision in decision block 264 is “YES” when the Care Giver “CG” needs to select a care provider. On the other hand, the decision in decision block 264 is “NO” when the Care Giver “CG” has already selected a care provider (e.g., in block 224 (see FIG. 3A-1)).

When the decision in decision block 264 is “YES,” the system 100 advances to decision block 266. When the decision in decision block 264 is “NO,” the system 100 advances to block 276 (discussed below).

In decision block 266, the system 100 determines whether the Care Giver “CG” will select a care provider using the method 330. The decision in decision block 266 is “YES” when the system 100 will perform the method 330. Otherwise, the decision in decision block 266 is “NO” when the system 100 will not perform the method 330. By way of a non-limiting example, the system 100 may decide to perform the method 330 when the Care Giver “CG” has indicated that the Care Giver “CG” is traveling.

When the decision in decision block 266 is “YES,” the method 330 is performed in block 268.

Because the Opinion Request may be canceled during the performance of the method 330, in decision block 270, the system 100 determines whether the Opinion Request has been canceled. If the Opinion Request has been canceled, the method 220 terminates. On the other hand, if the Opinion Request has not been canceled, the system 100 advances to block 276.

When the decision in decision block 266 is “NO,” in block 272, the system 100 instructs the non-expert user interface 120 to display a list of selectable Care Providers. FIG. 3F illustrates the non-expert user interface 120 configured to display a selectable button B-4 (for selecting a preferred care provider) and a selectable button B-5 (for selecting a care provider other than the preferred care provider). When the button B-5 is selected, the non-expert user interface 120 is configured to display a list of selectable Care Providers excluding the preferred care provider.

Returning to FIG. 3A-2, the information and details about each of the care providers may be displayed with the list help the Care Giver “CG” make an appropriate selection. The list may also display an indication of the availability of each of the care provider. By way of another example, the list may display response times for the care providers.

In block 274, the system 100 receives an identification of one or more of the Care Providers (in the Care Provider Network “CP NETWORK”) entered by the Care Giver “CG” into the non-expert user interface 120. For ease of illustration, the method 220 will be described with respect to the Care Giver “CG” having selected the Care Provider “CP” in block 274.

Then, in block 276, the non-expert user interface 120 submits the Opinion Request to the system 100 for processing in response to receiving a command from the Care Giver “CG” to submit the Opinion Request.

In optional decision block 278, the system 100 may determine whether the core system 114 stores any preprepared information (e.g., information provided by one or more third parties) related to the one or more Primary Complaints selected in block 230 and/or one or more of the responses received from the Care Giver “CG” in block 244. The decision in optional decision block 278 is “YES” when the core system 114 stores information related to one of the Primary Complaints selected in block 230 and/or one of the responses received in block 244. On the other hand, the decision in optional decision block 278 is “NO” when the core system 114 does not store information related to one of the Primary Complaints selected in block 230 or one of the responses received in block 244.

When the decision in optional decision block 278 is “YES,” in block 280, the core system 114 sends the preprepared information to the CG computing device 110 for display thereby to the Care Giver “CG.” Then, the system 100 advances to decision block 298.

When the decision in optional decision block 278 is “NO,” the system 100 advances to decision block 298.

In decision block 298, the system 100 receives an indication as to whether the Care Giver “CG” would like to receive an Expert Opinion from a care provider in the Care Provider Network “CP NETWORK” other than the one or more selected in block 274 (a Second Opinion). For example, the Care Giver “CG” may request an opinion from the Alternate Care Provider “ACP.” The decision in decision block 298 is “YES” when the Care Giver “CG” would like a second opinion. Otherwise, the decision in decision block 298 is “NO.”

When the decision in decision block 298 is “YES,” the system 100 performs the method 600 illustrated in FIG. 7A. Then, the method 220 terminates.

When the decision in decision block 298 is “NO,” the method 220 terminates.

After the Opinion Request has been submitted, the event created by the Opinion Request is referred to as a Live Case.

### Method 330: Select Care Provider(s)

FIG. 3B is a flow diagram illustrating the method 330 performed by the system 100 (illustrated in FIG. 1). The method 330 configures the non-expert user interface 120 for display on the CG computing device 110 and receives input from the Care Giver “CG.”

In first block 332, the system 100 constructs a list of preferred care providers and instructs the non-expert user interface 120 to display the list. The system 110 constructs the list based on an address stored in the health profile associated with the Care Recipient “CR.” The care providers of the list are selectable by the Care Giver “CG.” In block 332, the system 100 also instructs the non-expert user interface 120 to display a selectable option that the Care Giver “CG” may select to indicate that the Care Giver “CG” is traveling and therefore, not at the address stored in the health profile associated with the Care Recipient “CR.”

In decision block 334, the system 100 determines whether the Care Giver “CG” has selected the selectable option indicating the Care Giver “CG” is traveling or otherwise away from home. The decision in decision block 334 is “YES” when the Care Giver “CG” has selected the selectable option. Otherwise, the decision in decision block 334 is “NO.”

When the decision in decision block 334 is “NO,” the system 100 advances to decision block 336.

When the decision in decision block 334 is “YES,” the system 100 advances to decision block 338. In decision block 338, the system 100 instructs the non-expert user interface 120 to display a selectable option that the Care Giver “CG” may select to indicate that the Care Giver “CG” wishes to select one or more care providers based on the current location of the Care Giver “CG.” In decision block 338, the system 100 also determines whether the Care Giver “CG” has selected the selectable option indicating the Care Giver “CG” wishes to select one or more care providers based on the current location of the Care Giver “CG.” The decision in decision block 338 is “YES” when the Care Giver “CG” has selected the selectable option. Otherwise, the decision in decision block 338 is “NO.”

When the decision in decision block 338 is “NO,” the system 100 advances to decision block 336.

When the decision in decision block 338 is “YES,” in block 340, the system 100 obtains the current location of the Care Giver “CG.” The system 100 may obtain the current location of the Care Giver “CG” from a conventional geolocation module (not shown) installed in the CG computing device 110 illustrated in FIG. 1. By way of another non-limiting example, the system 100 may obtain the current location of the Care Giver “CG” from data entered manually by the Care Giver “CG” into the CG computing device 110. For example, the system 100 may instruct the non-expert user interface 120 to request that the Care Giver “CG” manually enter a current location.

In block 342, the system 100 constructs a new list of care providers based on current location of the Care Giver “CG” and instructs the non-expert user interface 120 to display the new list. The care providers displayed in the new list are selectable by the Care Giver “CG.” The new list includes care providers allowed to practice in the current location of the Care Giver “CG.”

In decision block 344, the system 100 determines whether the Care Giver “CG” has selected a care provider from the new list. The decision in decision block 344 is “YES” when the Care Giver “CG” has selected a care provider from the new list. Otherwise, the decision in decision block 344 is “NO” when the Care Giver “CG” has not selected a care provider from the new list or has indicated that the Care Giver “CG” does not wish to select a care provider from the new list.

When the decision in decision block 344 is “YES,” the method 330 terminates.

When the decision in decision block 344 is “NO,” in block 346, the system 100 instructs the non-expert user interface 120 to display a message informing the Care Giver “CG” that a care provider must be selected. Then, in block 348, the Opinion Request is canceled and the method 330 terminates.

In decision block 336, the system 100 determines whether the Care Giver “CG” has selected a care provider from the list of preferred care providers (displayed in block 332). The decision in decision block 336 is “YES” when the Care Giver “CG” has selected a care provider from the list of preferred care providers. Otherwise, the decision in decision block 336 is “NO” when the Care Giver “CG” has not selected a care provider from the list of preferred care providers.

When the decision in decision block 336 is “YES,” the method 330 terminates.

When the decision in decision block 336 is “NO,” in block 352, the system 100 constructs a list of care providers practicing in a preferred practice and instructs the non-expert user interface 120 to display this list. The care providers displayed in this list are selectable by the Care Giver “CG.” The preferred practice may be identified in the health profile associated with the Care Recipient “CR.”

In decision block 354, the system 100 determines whether the Care Giver “CG” has selected a care provider from the list of care providers practicing in a preferred practice (displayed in block 352). The decision in decision block 354 is “YES” when the Care Giver “CG” has selected a care provider from the list. Otherwise, the decision in decision block 354 is “NO” when the Care Giver “CG” has not selected a care provider from the list.

When the decision in decision block 354 is “YES,” the method 330 terminates.

When the decision in decision block 354 is “NO,” in block 356, the system 100 constructs a list of care providers allowed to practice at the location of the Care Giver “CG” (based on the address in the health profile associated with the Care Recipient “CR”) and instructs the non-expert user interface 120 to display this list. The care providers displayed in this list are selectable by the Care Giver “CG.”

In decision block 360, the system 100 determines whether the Care Giver “CG” has selected a care provider from the list of care providers allowed to practice at the location of the Care Giver “CG” (displayed in block 356). The decision in decision block 360 is “YES” when the Care Giver “CG” has selected a care provider from the list. Otherwise, the decision in decision block 360 is “NO” when the Care Giver “CG” has not selected a care provider from the list.

When the decision in decision block 360 is “YES,” the method 330 terminates.

When the decision in decision block 360 is “NO,” the system 100 advances to block 346.

### Method 300: Revise a Live Case

Turning to FIG. 4, the method 300 configures the non-expert user interface 120 for display on the CG computing device 110 and receives input from the Care Giver “CG.” The method 300 is performed after a new Opinion Request has been created using the method 220 (illustrated in FIGS. 3A-1 and 3A-2).

In first block 310, the system 100 receives an identification of a Live Case that the Care Giver “CG” would like to revise in some way (e.g., add additional information, ask another question, etc.).

In decision block 314, the system 100 determines whether the Live Case identified is being reviewed by the Care Provider “CP” for whom the Live Case was submitted. The decision in decision block 314 is “YES” when the Live Case identified is being reviewed. Otherwise, the decision in decision block 314 is “NO.”

When the decision in decision block 314 is “YES,” in block 318, the system 100 determines the Live Case is locked. Optionally, the non-expert user interface 120 may be instructed to display a message indicating the Live Case is locked. Then, the method 300 terminates.

When the decision in decision block 314 is “NO,” in block 320, the system 100 locks the Live Case so the Care Provider “CP” for whom the Live Case was submitted cannot access the Live Case.

Then, in block 324, the system 100 configures the non-expert user interface 120 to allow the Care Giver “CG” to enter additional information into the Live Case. Optionally, the Care Giver “CG” may be permitted to add new information but prevented from editing information added previously. Then, in block 328, the Live Case is submitted by the non-expert user interface 120 (in response to a command from the Care Giver “CG” to do so) to the core system 114. Then, the method 300 terminates.

### Method 400: Re-Route an Opinion Request to a Different Care Provider

Turning to FIG. 5, the method 400 configures the non-expert user interface 120 for display on the CG computing device 110 and receives input from the Care Giver “CG.” The method 400 is performed after a new Opinion Request has been created using the method 220 (illustrated in FIGS. 3A-1 and 3A-2).

In first block 410, the system 100 receives an identification of a Live Case that the Care Giver “CG” would like to re-route to a care provider other than the one for whom the Live Case was previously submitted.

In decision block 414, the system 100 determines whether the Live Case identified is being reviewed by the Care Provider “CP” for whom the Live Case was submitted. The decision in decision block 414 is “YES” when the Live Case identified is being reviewed. Otherwise, the decision in decision block 414 is “NO.”

When the decision in decision block 414 is “YES,” in block 418, the system 100 determines the Live Case is locked. Optionally, the non-expert user interface 120 may be instructed to display a message indicating the Live Case is locked. Then, the method 400 terminates.

When the decision in decision block 414 is “NO,” in block 430, the system 100 may lock the Live Case so the care provider(s) for whom the Live Case was submitted cannot access the Live Case. Then, the system 100 removes the care provider(s) previously identified from the Live Case.

Then, in block 440, the system 100 configures the non-expert user interface 120 to allow the Care Giver “CG” to select a different care provider(s) in the Care Provider Network “CP NETWORK” for the Live Case. Then, in block 442, the Live Case is submitted by the non-expert user interface 120 (in response to a command from the Care Giver “CG” to do so) to the core system 114. Then, the method 400 terminates.

### Method 500: Review an Expert Opinion

Turning to FIG. 6A, the method 500 configures the non-expert user interface 120 for display on the CG computing device 110 and receives input from the Care Giver “CG.” The method 500 is performed after a Care Provider “CP” has provided an Expert Opinion to be reviewed by the Care Giver “CG.” The method 500 is also performed after the non-expert user interface 120 has received a selection from the Care Giver “CG” of the “Review an Expert Opinion” option.

The Expert Opinion may have one or more of the following components:
1. a diagnosis;
2. a Management Protocol;
3. one or more follow up instructions; and
4. a referral to another care provider in the Care Provider Network “CP NETWORK” (e.g., a specialist).

In first block 505, the non-expert user interface 120 receives a command from the Care Giver “CG” to display at least a portion of the Expert Opinion. For example, in block 505, the non-expert user interface 120 may display the diagnosis and the Management Protocol. FIG. 6C illustrates the non-expert user interface 120 displaying at least a portion of the Expert Opinion. FIG. 6C illustrates a single page display (e.g., a screen of a mobile device) that may be used to display the non-expert user interface 120 to the Care Giver “CG” in the method 500.

In block 510, the system 100 identifies a set of actions not yet completed (“active”). The set of actions may be specified as needing to be taken within a window of time. As is apparent to those of ordinary skill in the art, the Management Protocol portion of the Expert Opinion may include several actions to be taken and completed within varying amounts of time. Thus, when the Care Provider “CP” enters the Medical Protocol(s) included in the Management Protocol, the Care Provider “CP” specifies a “complete by date/time.” For example, the Care Provider “CP” may specify that a particular mediation is to be taken twice a day. The complete by dates are used by the system 100 to group the actions into sets and display the actions within a group together. The sets may be ordered according to their respective complete by dates/times.

In block 520, the non-expert user interface 120 receives a selection of an action within the set.

In decision block 522, the non-expert user interface 120 receives an indication from the Care Giver “CG” as to whether the Care Giver “CG” would like to assign the action to another care giver in the Care Giver Network “CG NETWORK.” The decision in decision block 522 is “YES,” when the Care Giver “CG” would like to assign the action to another care giver in the Care Giver Network “CG NETWORK.” Otherwise, the decision in decision block 522 is “NO.”

When the decision in decision block 522 is “YES,” in block 540, the non-expert user interface 120 receives an assignment from the Care Giver “CG” of the action to another care giver in the Care Giver Network “CG NETWORK.” Then, the system 100 advances to decision block 535. Optionally, the system 100 may send a notification to the computing device operated by the care giver to whom the action has been assigned.

When the decision in decision block 522 is “NO,” after the Care Giver “CG” performs an action in the set of actions, in block 530, the Care Giver “CG” confirms the completion of the action with the system 100. Thus, in block 530, the non-expert user interface 120 receives an indication from the Care Giver “CG” that an action has been completed.

Then, in decision block 535, the system 100 determines whether all of the actions of the set of actions have been completed. If all of the actions of the set of actions have not been completed, the decision in decision block 535 is “YES” and the system 100 returns to block 510.

If all of the actions of the set of actions have been completed, the decision in decision block 535 is “NO,” and the method 500 terminates.

FIG. 6B illustrates a method 550 of tracking the compliance or adherence of the Care Giver “CG” to the Medical Protocol(s) included in the Management Protocol portion of the Expert Opinion. In decision block 560, the system 100 determines whether the Care Giver “CG” has failed to confirm (in block 530 of the method 500 illustrated in FIG. 6A) that one or more actions have been taken by their complete by dates/times specified by the Care Provider “CP” for the actions.

The decision in decision block 560 is “NO” when the system 100 determines that the Care Giver “CG” has failed to confirm that one or more actions have been taken by their complete by dates/times specified by the Care Provider “CP” for the actions. Otherwise, the decision in decision block 560 is “YES.”

When the decision in decision block 560 is “NO,” in block 562, the system 100 sends a notification to the CG computing device indicating that one or more actions is overdue. Then, the method 550 terminates.

When the decision in decision block 560 is “YES,” the method 550 terminates.

### Method 600: Request a Second or Specialist Opinion

Turning to FIG. 7A, the method 600 configures the non-expert user interface 120 for display on the CG computing device 110 and receives input from the Care Giver “CG.” The method 600 is performed during the creation of a new Opinion Request or after one has been created using the method 220 (illustrated in FIGS. 3A-1 and 3A-2).

In first decision block 610, the system 100 receives an indication from the non-expert user interface 120 (entered by the Care Giver “CG”) indicating whether the Second or Specialist Opinion relates to a previously created Live Case or is a new Opinion Request. The decision in decision block 610 is “NEW” when the Second or Specialist Opinion relates to a new Opinion Request. Otherwise, the decision in decision block 610 is “EXISTING” when the Second or Specialist Opinion relates to a previously created Live Case.

When the decision in decision block 610 is “NEW,” the system 100 performs the method 220 illustrated in FIGS. 3A-1 and 3A-2.

When the decision in decision block 610 is “EXISTING,” the system 100 advances to decision block 620.

In decision block 620, the system 100 determines whether the Second or Specialist Opinion was a referral from or recommendation by the Care Provider “CP” specified for the Opinion Request. The decision in decision block 620 is “YES” when the Second or Specialist Opinion is a referral. Otherwise, the decision in decision block 620 is “NO” when the Second or Specialist Opinion is not a referral.

When the decision block 620 is “YES,” in block 622, the non-expert user interface 120 receives instructions from the Care Giver “CG” to route the Live Case to the care provider (e.g., the Alternate Care Provider “ACP”) identified by the Care Provider “CP” in the referral information portion of the Expert Opinion.

Then, in block 624, the non-expert user interface 120 receives instructions from the Care Giver “CG” to submit the Live Case to the core system 114 for processing and routing. The Live Case may be locked with respect to the care provider originally assigned to the Live Case. Then, the method 700 may be performed.

The system 100 routes the Live Case to the care provider to which the Live Case has been referred. The routed Live Case may include the diagnosis portion of the Expert Opinion, the Management Protocol portion of the Expert Opinion, and the referral information portion of the Expert Opinion (which may include notes for the care provider to which the Live Case has been referred).

When the decision block 620 is “NO,” in block 630, the non-expert user interface 120 receives an identification of one or more of the care providers of the Care Provider Network “CP NETWORK” to which to route the Live Case for a Second Opinion and/or a Specialist Opinion.

In decision block 632, the non-expert user interface 120 receives an indication whether to include portions of the Expert Opinion in the Live Case to be routed to the care provider(s) identified in block 630. For example, in decision block 632, the Care Giver “CG” may decide whether to include the diagnosis portion of the Expert Opinion, and/or the Management Protocol portion of the Expert Opinion in the Live Case routed to the care provider(s) identified in block 630.

The decision in decision block 632 is “YES” when the indication received in block 632 indicates to include portions of the Expert Opinion in the Live Case to be routed to the care provider(s) identified in block 630. Otherwise, the decision in decision block 632 is “NO” when the indication received in block 632 indicates not to include portions of the Expert Opinion in the Live Case to be routed to the care provider(s) identified in block 630.

When the decision in decision block 632 is “YES,” the system 100 advances to block 622.

When the decision in decision block 632 is “NO,” in block 636, the system 100 clones the Live Case to create a duplicate Live Case. The duplicate Live Case is linked to (or otherwise associated with) the original Live Case. Then, in block 640, the non-expert user interface 120 receives instructions from the Care Giver “CG” to submit either the Live Case or the duplicate Live Case to the core system 114 for processing and routing. The submitted Live Case may be locked with respect to the care provider originally assigned to the Live Case. Then, the method 700 may be performed.

### Method 650: Edit Health Profile

Turning to FIG. 7B, the method 650 configures the non-expert user interface 120 for display on the CG computing device 110 and receives input from the Care Giver “CG.” The method 650 updates the Health Profile associated with the Care Recipient “CR” and may be performed at any time. The health profile is auto-updating with each Live Case associated with the Care Recipient “CR” to include information relevant to the Live Case. The method 650 is performed after the non-expert user interface 120 has received a selection from the Care Giver “CG” of the “Edit Health Profile” option. For example, the non-expert user interface 120 may receive the selection of the “Edit Health Profile” option from the Care Giver “CG.”

In block 656, the non-expert user interface 120 displays at least the following options to the Care Giver “CG:”
1. New Measurement Captures;
2. New Developmental Observation;
3. New Journal Entry; and
4. New Journal

In block 660, the system 100 receives a selection from the Care Giver “CG” of one of the above options via the non-expert user interface 120. By way of an example, the non-expert user interface 120 may receive a selection of the “New Measurement Captures” option.

If the Care Giver “CG” selected “New Measurement Captures,” in block 662, the system 100 configures the non-expert user interface 120 to receive measurement information (e.g., physical measurements such as height, weight, and the like) entered by the Care Giver “CG” into the non-expert user interface 120.

After the Care Giver “CG” has entered measurement information into the non-expert user interface 120, in block 664, the system 100 updates the Health Profile associated with the Care Recipient “CR” based on the information entered or overrides the request and updates the Health Profile later (Height/Weight will be requested if medication or metric dependant management protocol is being delivered). Then, in block 668, the system 100 updates a Historical Plot or View (e.g., graphs 667A and 667B) of the measurement information associated with the Care Recipient “CR” based on the information entered. The Historical Plot or View displays the measurement information over time to graphically depict changes in the measurement information. Then, the system advances to decision block 670.

When the Care Giver “CG” has selected the “New Developmental Observation” option, in block 672, the system 100 configures the non-expert user interface 120 to display observations expected for Care Recipient “CR” based on information (e.g., age, gender, and the like) stored in the Health Profile associated with the Care Recipient “CR.” In block 674, the non-expert user interface 120 receives information from the Care Giver “CG” validating the status of the Care Recipient “CR” with respect to the observations. In the FIG. 7C, the non-expert user interface 120 is illustrated displaying inputs (illustrated as “thumbs up” and “thumbs down” symbols) for receiving observations related to the Care Recipient “CR.” In block 676, the system 100 updates the Health Profile associated with the Care Recipient “CR” based on the information entered. Then, in block 678, the system 100 updates a Developmental Report View. In the FIG. 7D, the non-expert user interface 120 is illustrated displaying an exemplary Developmental Report View. Returning to FIG. 7B, then, the system advances to decision block 670.

When the Care Giver “CG” selects “New Journal Entry,” in block 680, the system 100 configures the non-expert user interface 120 to display a graphical scale having a plurality of selectable values and receive a selection from the Care Giver “CG” selecting one of the values of the graphical scale. For example, FIG. 7E illustrates the non-expert user interface 120 displaying a graphical scale S-1 having a plurality of selectable symbols (illustrated as cartoon faces). Returning to FIG. 7B, then, in block 682, the system 100 updates the Health Profile associated with the Care Recipient “CR” based on the value selected in block 680. Then, in block 668, the system 100 updates a Journal Summary View. FIG. 7F illustrates the non-expert user interface 120 displaying a Journal Summary View, which includes a graph of the data entered. Returning to FIG. 7B, then, the system advances to decision block 670.

If the Care Giver “CG” selected “New Journal,” in block 688, the system 100 configures the non-expert user interface 120 to display a plurality of selectable journal templates and receive selections of the journal templates from the Care Giver “CG.” In block 690, the non-expert user interface 120 receives details (e.g., a journal name, journal type, and the like) related to the new journal entered by the Care Giver “CG.” In block 692, the system 100 configures the non-expert user interface 120 to display the graphical scale (described above) and receive a selection from the Care Giver “CG” selecting one of the values of the graphical scale. Then, in block 694, the system 100 updates the Health Profile associated with the Care Recipient “CR” based on the value selected in block 692. Then, in block 696, the system 100 updates the Journal Summary View (described above and depicted in FIG. 7F). Then, the system advances to decision block 670.

In decision block 670, if the Care Giver “CG” has finished selecting options displayed by the non-expert user interface 120 (i.e., the decision in decision block 670 is “YES”), the method 650 terminates. Otherwise, if the Care Giver “CG” has not finished selecting options displayed by the non-expert user interface 120 (i.e., the decision in decision block 670 is “NO”), the method 650 returns to block 656 whereat the options are displayed to the Care Giver “CG” for selection thereby.

### Method 700: Create Intelligent Resident Note

After the method 220, has been performed, the Live Case has been submitted to the system 100. The system 100 configures the information contained in the Live Case for display by the expert user interface 122. For example, the system 100 may translate the information of the Live Case into an automatically generated Intelligent Resident Note (or an expert systems summary note). Thus, the system 100 may be characterized as converting the plain-language description of the issue (accepted by the Care Giver “CG” in decision block 260 of the method 220 illustrated in FIGS. 3A-1 and 3A-2into an expert format using expert language for review by the Care Provider “CP.”

The method 700 creates an Intelligent Resident Note from the information contained in the Live Case. The Intelligent Resident Note presents information in a format and order that is acceptable, efficient, and readily reviewable by the Care Provider “CP” as a “normal” presentation of such information.

The Intelligent Resident Note may include the following:
1. Subjective Information obtained from the tailored health interrogation process 245 (e.g., via blocks 242, 244, and 246 of the method 220 illustrated in FIG. 3A-1, characterization of the primary complaint, and associated issues converted into expert language and presented in an expert format;
2. Objective Information, such as vital signs, other observational information, and other measurement data (e.g., height, weight, developmental milestones, temperature, oximetry, respiratory rate, pulse rate, and the like), displayed as static data in an appropriate format (e.g., list format, streaming format, chart format, combinations thereof, and the like);
3. Assessment Information provided by the Care Provider “CP,” such analysis and differential diagnosis (requested from the Care Giver “CG”) that allows the development of the Management Protocol with appropriate medical protocols; and
4. Plan Information provided by Care Provider “CP” that includes the Management Protocol, prescription information, and delivery information.

In first block 710, the Live Case is stored in a memory location accessible by the core system 114.

In next block 720, the system 100 obtains additional health data associated with the Care Recipient “CR.” The additional health data may include information stored in the health profile associated with the Care Recipient “CR.”

Then, in block 730, the system 100 automatically generates (or builds) the portions of the Intelligent Resident Note not provided by the Care Provider “CP” based on the information contained in the Live Case and the additional health data. The system 100 includes a dictionary of known collective plain language terms associated with expert terminology. The dictionary may be populated over time by data-mining terms and phrases from Opinion Requests and associating those plain language terms with expert terminology mined from Expert Opinions provided in response to the Opinion Requests. Thus, a data dictionary may be constructed and used by the system 100 to convert between plain language descriptions and Intelligent Resident Notes. The Intelligent Resident Note is stored and associated with the Live Case for which the Intelligent Resident Note was created.

In optional block 740, the care provider(s) assigned to the Live Case are notified of the assignment of the new Live Case.

The system 100 may be configured to monitor the Intelligent Resident Note to determine whether it has been reviewed within a predetermined amount of time. The predetermined amount of time may be set by the Care Giver “CG,” the system 100, the Care Provider “CP,” an organization associated with any of the forgoing, and the like.

After the predetermined amount of time has elapsed, in decision block 750, the system 100 determines whether the Intelligent Resident Note has been reviewed. The decision in decision block 750 is “YES” when the Intelligent Resident Note has not been reviewed within the predetermined amount of time. An amount of time that elapsed between the submission of the Opinion Request and the submission of the Expert Opinion may be displayed to the Care Giver “CG” in the non-expert user interface 120 to provide transparency related to the review time. Otherwise, the decision in decision block 750 is “NO” when the Intelligent Resident Note has been reviewed within the predetermined amount of time.

When the decision in decision block 750 is “NO,” the method 700 terminates. On the other hand, when the decision in decision block 750 is “YES,” in block 760, the system sends a reminder to the Care Provider “CP.” Optionally, the system 100 may send a notification to the Care Giver “CG” via the non-expert user interface 120 so that the Care Giver “CG” may instruct the system 100 to perform the method 400 (illustrated in FIG. 5) and use the non-expert user interface 122 to re-route the Live Case to a different care provider. Then, the method 700 terminates.

FIG. 9B-1 and 9B-2 illustrate an example of a single page display (e.g., a screen of a mobile device) that may be used to display the Intelligent Resident Note (in the expert user interface 122) to the Care Provider “CP.” The expert user interface 122 may be configured to allow scrolling along the single page display.

### Method 800

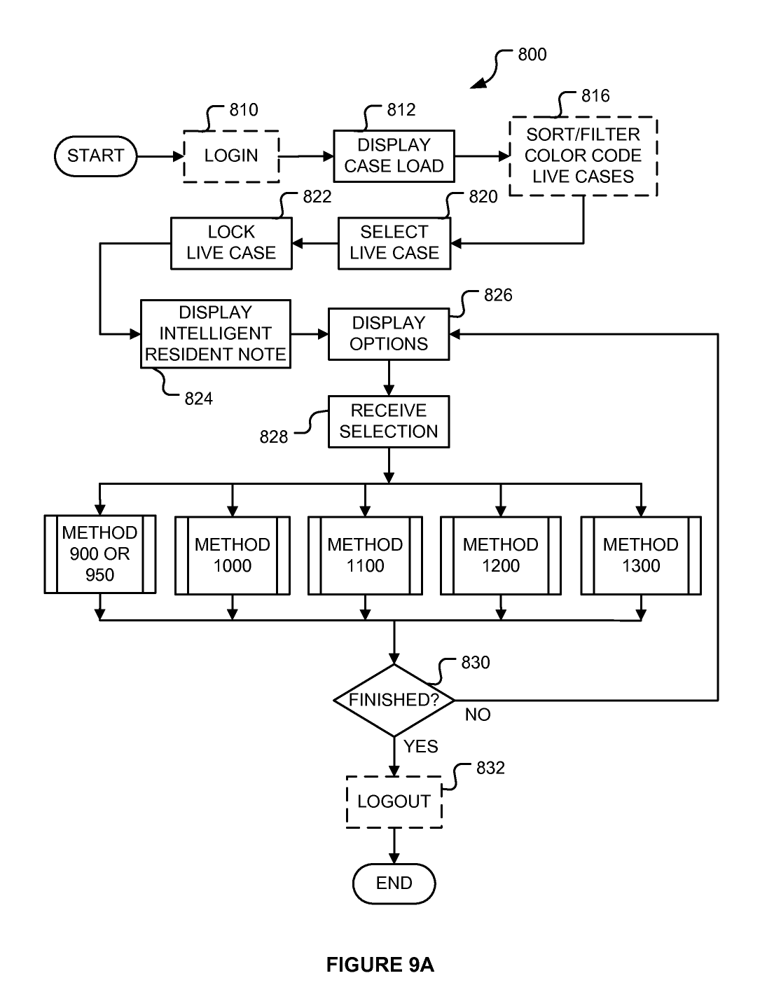

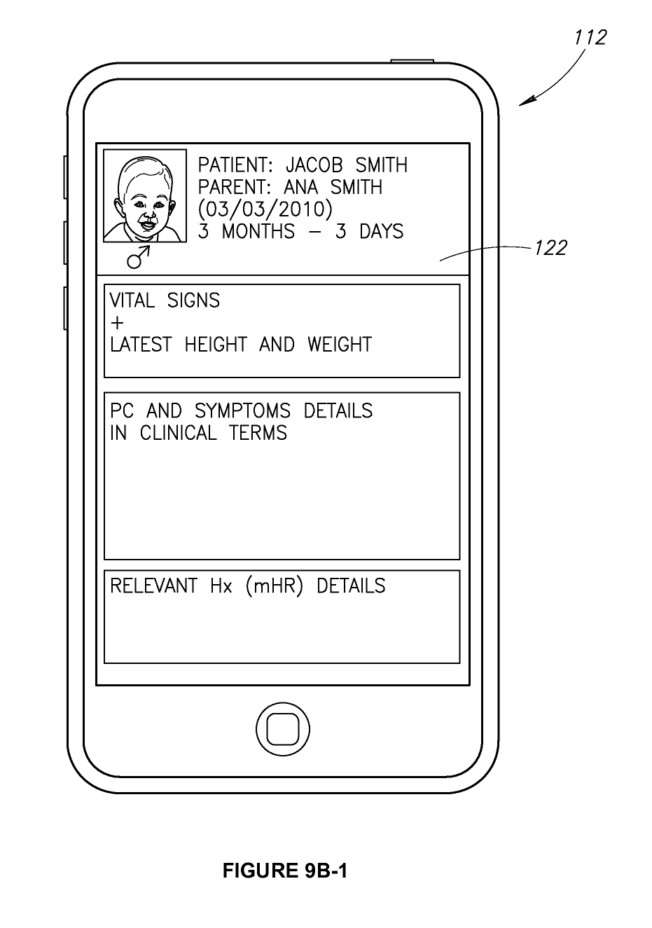

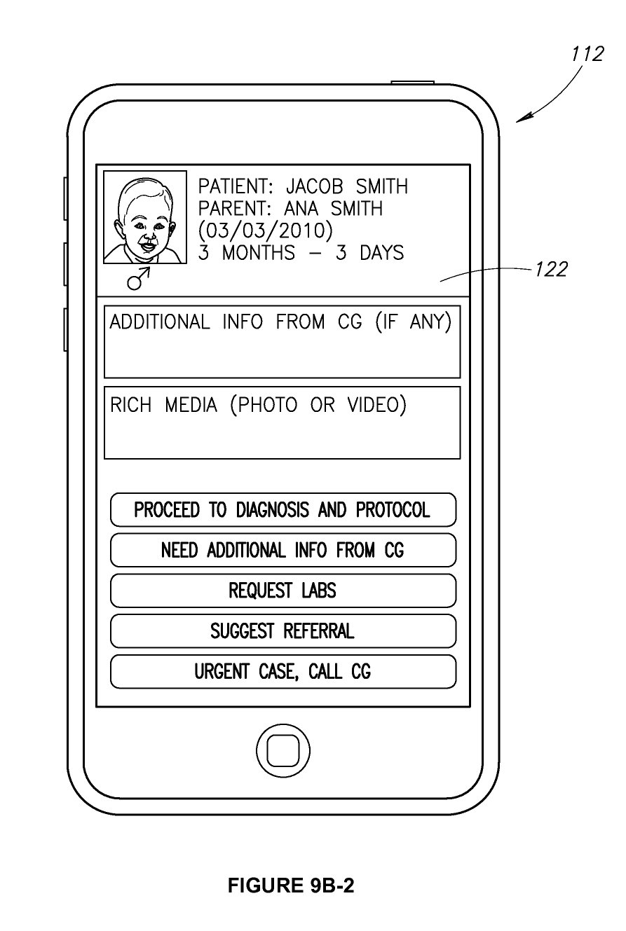

Turning to FIG. 9A, the method 800 configures the expert user interface 122 for display on the CP computing device 112 and receives input from the Care Provider “CP.” The method 800 configures the expert user interface 122 for display on the CP computing device 112 and receives input from the Care Provider “CP.”

In optional first block 810, the system 100 receives valid login information from the Care Provider “CP” and in response, displays the expert user interface 122 to the Care Provider “CP.” The present disclosure is not limited to use with any particular method of logging into the system 100. After the Care Provider “CP” has successfully logged into the core system 114, the expert user interface 122 may display a home page or greeting screen.

Returning to FIG. 9A, in block 812, the expert user interface 122 displays a “case load” to the Care Provider “CP.” The case load may be a list of Live Cases awaiting an Expert Opinion from the Care Provider “CP.” The Live Cases may be obtained by querying a Live Case database (described below) for Live Cases assigned to the Care Provider “CP” for which an Expert Opinion has not been submitted.

Returning to FIG. 9A, in optional block 816, the expert user interface 122 performs commands on the list provided by the Care Provider “CP.” For example, in block 816, the expert user interface 122 may sort, filter, and/or color code the Live Cases in the list. The commands may help the Care Provider “CP” perform triage on the list of Live Cases.

In block 820, the expert user interface 122 receives an identification (or selection) of one of the Live Cases in the list. In other words, in block 820, the Care Provider “CP” selects one of the Live Cases in the list and enters this selection into the expert user interface 122.

In block 822, the system 100 locks the Live Case selected in block 820 to prevent further editing of the Live Case by the Care Giver “CG.”

In block 824, the system 100 obtains the Intelligent Resident Note created by the method 700 (illustrated in FIG. 8) for the Live Case identified in block 820 and instructs the expert user interface 122 to display the Intelligent Resident Note. FIGS. 9B-1 and 9B-2 depict the expert user interface 122 displaying an exemplary Intelligent Resident Note.

Optionally, in block 824, the system 100 may also “surface” other potentially relevant information. For example, the system 100 may search a medical history associated with the Care Recipient “CR” for past events (e.g., past Live Cases) and data related to the one or more Primary Complaints and the information (responses provided during the tailored health investigation process 245 illustrated in FIG. 3A-1) provided by the Care Giver “CG” for the present Live Case. Information located by the search may be highlighted or otherwise brought to the attention of the Care Provider “CP” by the expert user interface 122. In this manner, while all data related to the Care Recipient “CR” may be available to the Care Provider “CP” when evaluating the Live Case, information considered potentially relevant may be more readily located and evaluated. A link to the medical history of the Care Recipient “CR” may also be displayed by the expert user interface 122.

Returning to FIG. 9A, in block 826, the expert user interface 122 displays at least the following options to the Care Provider “CP:”
1. Proceed to Medical Protocol;
2. Request additional details;
3. Suggest Investigation(s)/Test(s);
4. Suggest Specialist Review; and
5. Urgent case—call Care Giver “CG.”

FIG. 9B-2 depicts the expert user interface 122 displaying the above options.

Returning to FIG. 9A, in block 828, the system 100 receives a selection from the Care Provider “CP” of one of the above options.

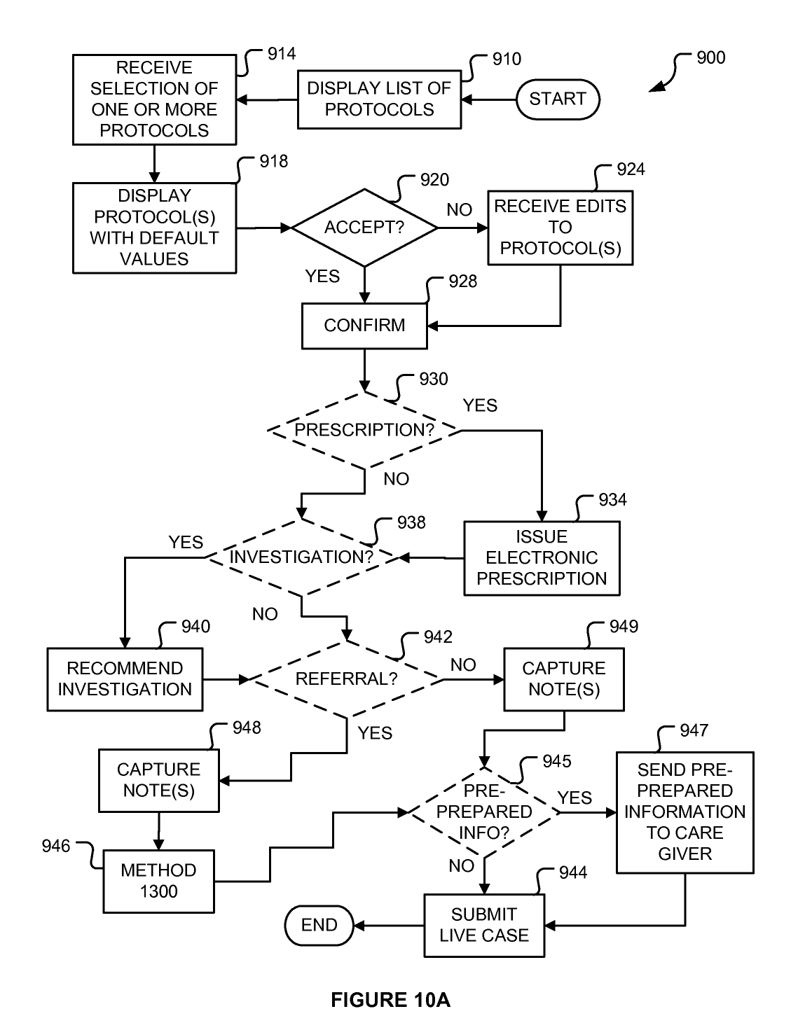

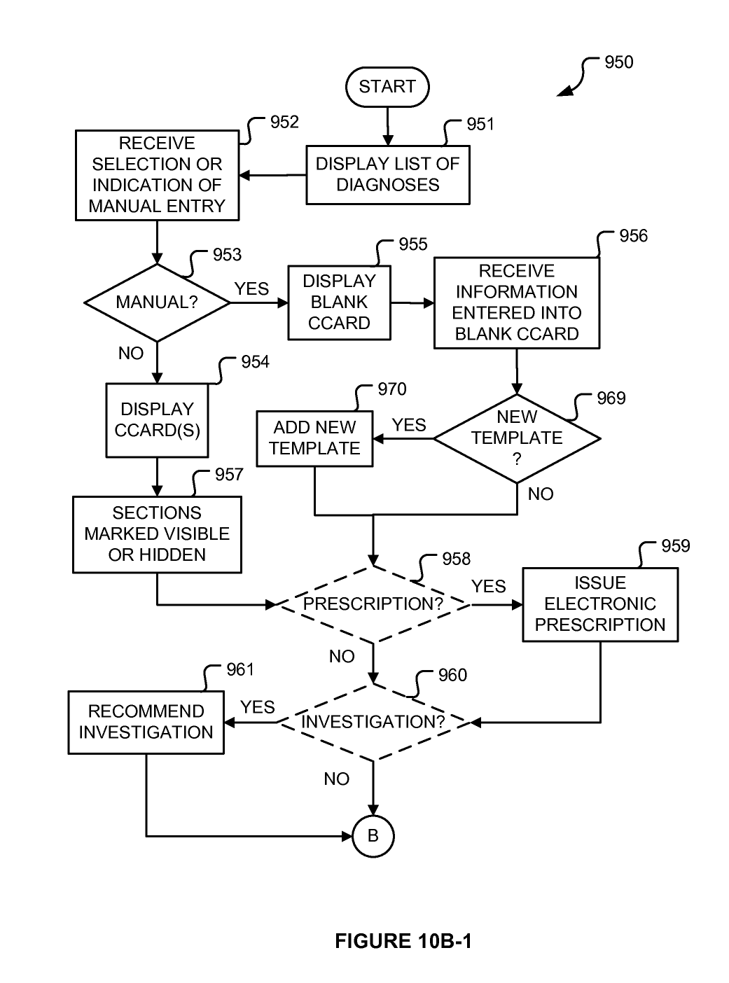

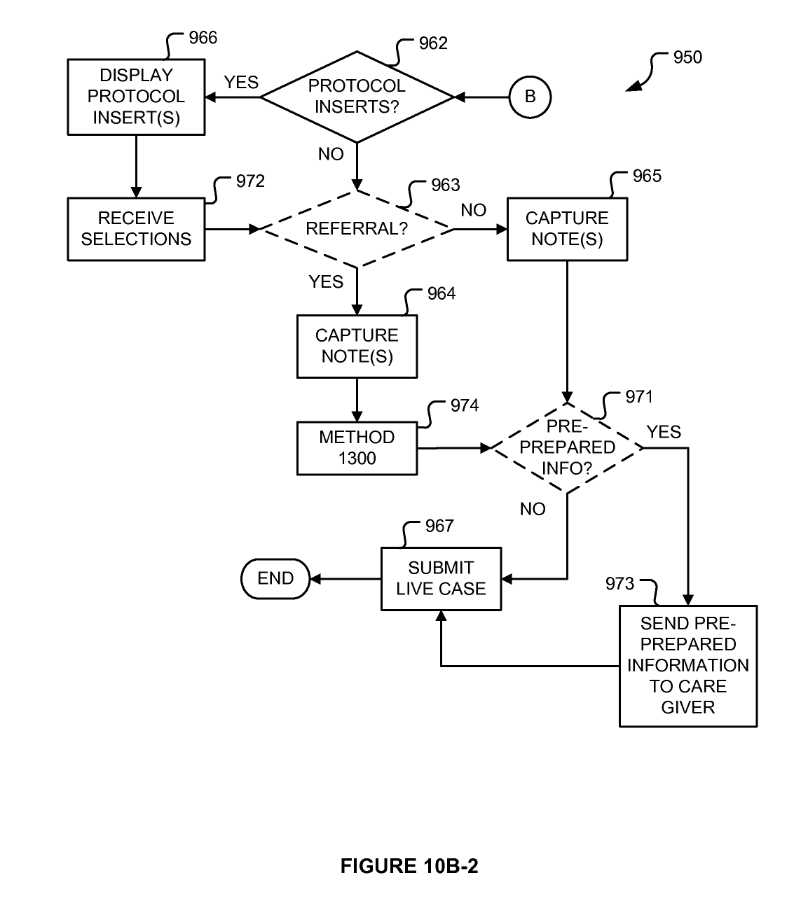

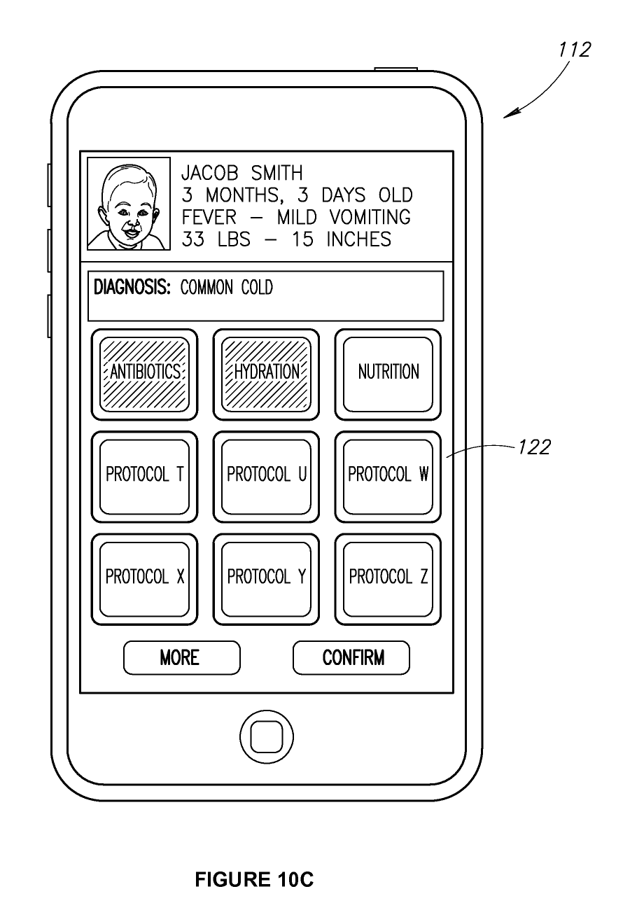

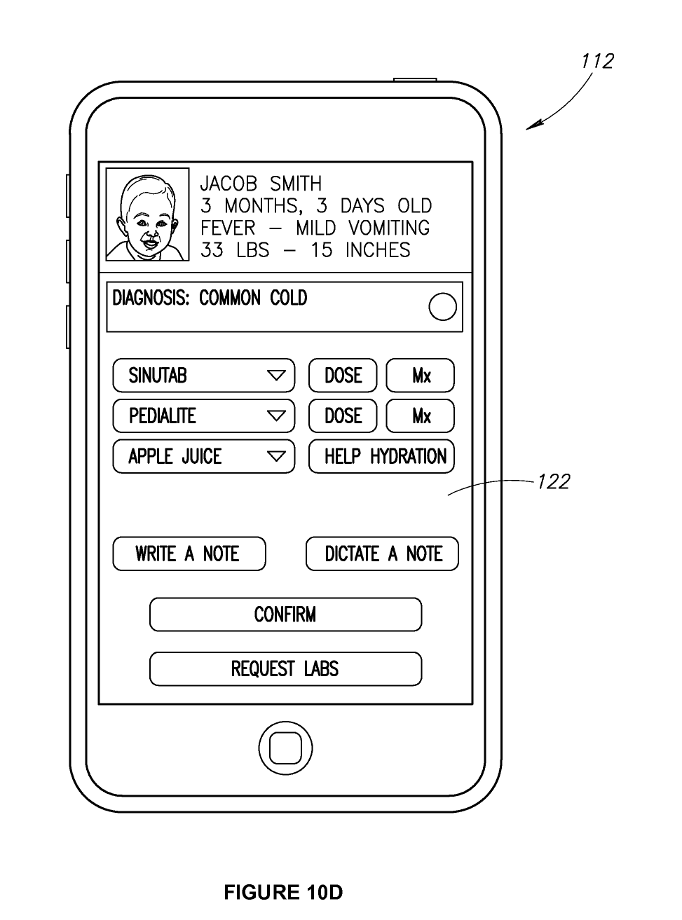

If the Care Provider “CP” selected “Proceed to Medical Protocol,” either a method 900 illustrated in FIG. 10A or a method 950 illustrated in FIGS. 10B-1 and 10B-2 is performed. Then, the system 100 advances to decision block 830.

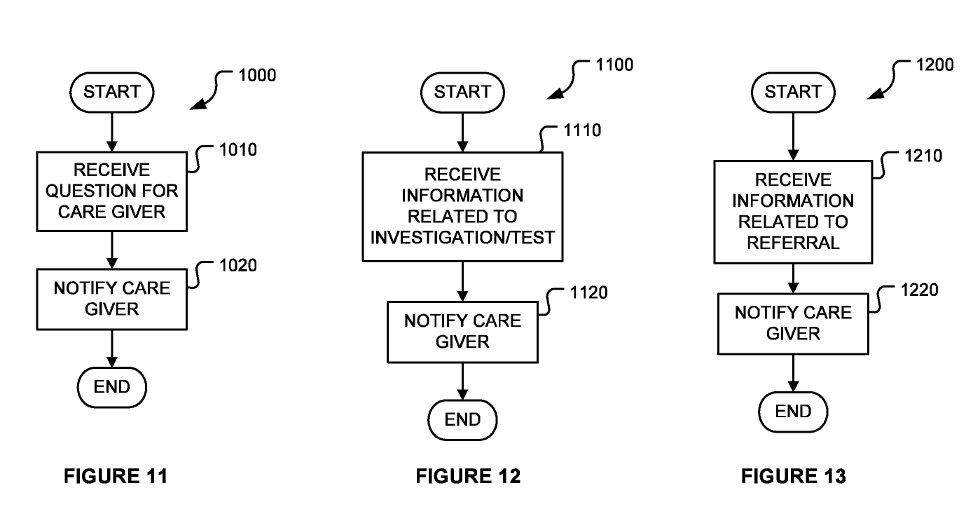

If the Care Provider “CP” selected “Request additional details,” a method 1000 illustrated in FIG. 11 is performed. Then, the system 100 advances to decision block 830.

If the Care Provider “CP” selected “Suggest Investigation(s)/Test(s),” a method 1100 illustrated in FIG. 12 is performed. Then, the system 100 advances to decision block 830.

If the Care Provider “CP” selected “Suggest Specialist Review,” a method 1200 illustrated in FIG. 13 is performed. Then, the system 100 advances to decision block 830.

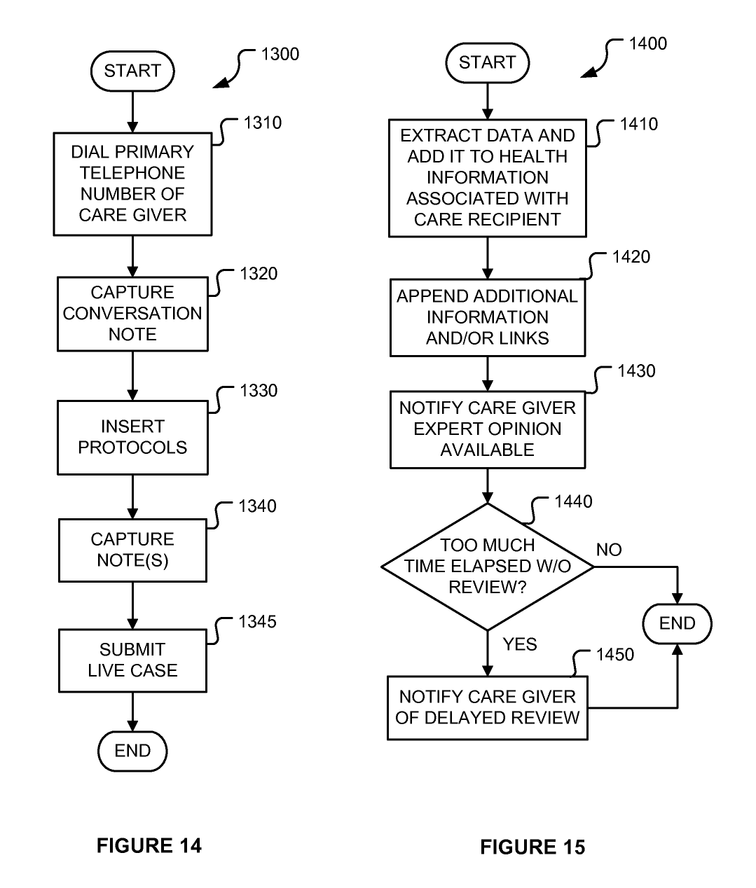

If the Care Provider “CP” selected “Urgent case—call Care Giver,” a method 1300 illustrated in FIG. 14 is performed. Then, the system 100 advances to decision block 830.

The decision in decision block 830 is “YES” when the Care Provider “CP” has finished selecting options displayed by the expert user interface 122. When the decision in decision block 830 is “YES,” the Care Provider “CP” may logout in optional block 832 and the method 800 terminates. The decision in decision block 830 is “NO” when the Care Provider “CP” has not finished selecting options displayed by the expert user interface 122. When the decision in decision block 830 is “NO,” the method 800 returns to block 826 whereat the options are displayed to the Care Provider “CP” for selection thereby.

### Method 900: First Embodiment: Proceed to Medical Protocol

FIG. 10A is a flow diagram illustrating the method 900, which is a first embodiment of a method of providing a medical protocol in response to an Opinion Request. The method 900 configures the expert user interface 122 for display on the CP computing device 112 and receives input from the Care Provider “CP.”

Before first block 910, the Care Provider “CP” may enter one or more diagnosis into the expert user interface 122. FIG. 10C depicts the expert user interface 122 after having received a diagnosis (e.g., “common cold”). The diagnosis may be the same as or different from the one or more Primary Complaints identified in the Live Case (e.g., the one or more Primary Complaints selected in block 230 of the method 220 illustrated in FIG. 3A-1).

Returning to FIG. 10A, in first block 910, the system 100 identifies one or more medical protocols associated with each of the one or more diagnosis provided by the Care Provider “CP” or other relevant protocol driving pieces of information (or data points), such as symptoms defined in Primary Complaint and provided by the Care Giver “CG.” For each diagnosis or relevant piece of information, the system 100 may identify the one or more medical protocols associated with the diagnosis by querying a medical protocol database (described below) for the diagnosis. Then, for each diagnosis, the medical protocols identified are displayed to the Care Provider “CP” via the expert user interface 122. FIG. 10C depicts the expert user interface 122 displaying multiple selectable medical protocols associated with the diagnosis. FIG. 10C depicts the expert user interface 122 after having received selections of “antibiotics” and “hydration” medical protocols from the Care Provider “CP.”

Returning to FIG. 10A, in next block 914, the expert user interface 122 receives a selection of one or more medical protocols for each of the diagnosis.

In next block 918, the system 100 instructs the expert user interface 122 to display the medical protocol(s) selected in block 914 with default values (e.g., default dosages) and/or default information (e.g., default instructions). FIG. 10D depicts the expert user interface 122 displaying default values and information associated with a selected medical protocol.

Returning to FIG. 10A, in decision block 920, the system 100 determines whether the Care Provider “CP” has indicated (by a command entered into the expert user interface 122), whether the default values and/or information are acceptable. The decision in decision block 920 is “YES” when the Care Provider “CP” has indicated that the default values and/or information are acceptable. Otherwise, the decision in decision block 920 is “NO.” In decision block 920, the expert user interface 122 may display a question asking the Care Provider “CP” whether the Care Provider “CP” accepts the default values and/or information.

When the decision in decision block 920 is “NO,” in block 924, the system 100 configures the expert user interface 122 to accept modifications entered by the Care Provider “CP” to the default values and/or information. Then, the system 100 advances to block 928.

When the decision in decision block 920 is “YES,” the system 100 advances to block 928.

In block 928, the system 100 receives an indication that the user has confirmed the medical protocols selected in block 914 via the expert user interface 122.

Optionally, the Care Provider “CP” may indicate the Care Provider “CP” would like to issue one or more prescriptions. In optional decision block 930, the system 100 determines whether the Care Provider “CP” has indicated (by a command entered into the expert user interface 122) the Care Provider “CP” would like to issue one or more prescriptions. The decision in decision block 930 is “YES” when the Care Provider “CP” has indicated that the Care Provider “CP” would like to issue one or more prescriptions. Otherwise, the decision in decision block 930 is “NO.” In decision block 930, the expert user interface 122 may display a question asking the Care Provider “CP” whether the Care Provider “CP” would like to issue one or more prescriptions.

When the decision in decision block 930 is “YES,” in block 934, the system 100 configures the expert user interface 122 to receive prescription information that may be used to generate one or more electronic prescriptions (eprescriptions) that may be filled by a pharmacy. Then, the system 100 advances to optional decision block 938.

When the decision in decision block 930 is “NO,” the system 100 advances to optional decision block 938.

Optionally, the Care Provider “CP” may indicate the Care Provider “CP” would like to recommend or order one or more investigations (e.g., laboratory tests). In optional decision block 938, the system 100 determines whether the Care Provider “CP” has indicated (by a command entered into the expert user interface 122) the Care Provider “CP” would like to recommend one or more investigations. When an investigation is recommended, the system 100 may help the Care Giver “CG” find the location whereat the investigation will be conducted. The system may also help locate where investigations can be performed or from which vendors an electronic order for an investigation can be placed. The system 100 may be configured to place such electronic orders. The system 100 may include geolocation capabilities that may be used to help choose vendors based on their proximity to the Care Giver “CG” and/or the Care Provider “CP.” Thus, wherever there is a service location, the system 100 may use geolocation as a basis for selection and providing selectable options. The decision in decision block 938 is “YES” when the Care Provider “CP” has indicated that the Care Provider “CP” would like to recommend one or more investigations. Otherwise, the decision in decision block 938 is “NO.” In decision block 938, the expert user interface 122 may display a question asking the Care Provider “CP” whether the Care Provider “CP” would like to recommend one or more investigations.

When the decision in decision block 938 is “YES,” in block 940, the system 100 configures the expert user interface 122 to receive recommendations for one or more investigations that may be used to conduct such investigations (einvestigations). Optionally, in block 940, the system 100 may perform the method 1100. Then, the system 100 advances to optional decision block 942.

When the decision in decision block 938 is “NO,” the system 100 advances to optional decision block 942.

Optionally, the Care Provider “CP” may indicate the Care Provider “CP” would like to recommend a referral to a different care provider (e.g., to a specialist). In optional decision block 942, the system 100 determines whether the Care Provider “CP” has indicated (by a command entered into the expert user interface 122) the Care Provider “CP” would like to recommend a referral to a different care provider (e.g., a specialist). The decision in decision block 942 is “YES” when the Care Provider “CP” has indicated that the Care Provider “CP” would like to recommend a referral to a different care provider. Otherwise, the decision in decision block 942 is “NO.” In decision block 942, the expert user interface 122 may display a question asking the Care Provider “CP” whether the Care Provider “CP” would like to recommend a referral to a different care provider.

When the decision in decision block 942 is “YES,” in block 948, the system 100 instructs the expert user interface 122 to display a free form text box into which the Care Provider “CP” may enter additional notes. The note(s) entered may be for the Care Giver “CG.” If any notes are entered by the Care Provider “CP,” in block 948, these notes are captured by the system 100. Next, in block 946, the system 100 performs the method 1300 illustrated in FIG. 14. Then, the system 100 advances to optional decision block 945.

When the decision in decision block 942 is “NO,” in block 949, the system 100 instructs the expert user interface 122 to display a free form text box into which the Care Provider “CP” may enter additional notes. The note(s) entered may be for the Care Giver “CG.” If any notes are entered by the Care Provider “CP,” in block 949, these notes are captured by the system 100. Then, the system 100 advances to optional decision block 945.

In optional decision block 945, the system 100 may determine whether the core system 114 stores any preprepared information (e.g., information provided by one or more third parties) related to the one or more diagnoses and/or medical protocols. The decision in optional decision block 945 is “YES” when the core system 114 stores information related to the one or more diagnoses and/or medical protocols. On the other hand, the decision in optional decision block 945 is “NO” when the core system 114 does not store information related to the one or more diagnoses and/or medical protocols.

When the decision in optional decision block 945 is “YES,” in block 947, the core system 114 sends the preprepared information to the CG computing device 110 for display thereby to the Care Giver “CG.” Then, the system 100 advances to block 944.

When the decision in optional decision block 945 is “NO,” the system 100 advances to block 944.

At this point, the Care Provider “CP” has finished entering the information for the Expert Opinion into the expert user interface 122. In block 944, the expert user interface 122 receives a command from the Care Provider “CP” to submit the information for the Expert Opinion. The expert user interface 122 submits the information for the Expert Opinion to the system 100 for processing in response to receiving the command from the Care Provider “CP” to submit the information for the Expert Opinion. Then, the method 900 terminates.

After the method 900 terminates, the Expert Opinion has been submitted to the system 100 for processing thereby. For example, a method 1400 (described below and illustrated in FIG. 15) may be performed by the core system 114 after the completion of the method 900.

### Method 950: Second Embodiment: Proceed to Medical Protocol

FIGS. 10B-1 and 10B-2 provide a flow diagram illustrating the method 950, which is a second embodiment of a method of providing a medical protocol in response to an Opinion Request. The method 950 configures the expert user interface 122 for display on the CP computing device 112 and receives input from the Care Provider “CP.”

Turning to FIG. 10B-1, in first block 951, the system 100 constructs a list of diagnoses and instructs the expert user interface 122 to display the list. The diagnoses displayed are selectable by the Care Provider “CP.” The list may include all possible diagnoses known to the core system 114 (e.g., diagnoses stored in a database) to avoid influencing the decision of the Care Provider “CP” when determining a diagnosis. The list of diagnoses may be sorted alphabetically to help the Care Provider “CP” locate a particular diagnosis in the list. The system 100 also instructs the expert user interface 122 to display a selectable option indicating the Care Provider “CP” would like to enter a diagnosis manually.

In block 952, the system 100 receives either an indication that the Care Provider “CP” has selected one or more of the diagnoses in the list or has selected the selectable option indicating the Care Provider “CP” would like to manually enter a diagnosis.

In decision block 953, the system 100 determines whether the Care Provider “CP” has selected the selectable option indicating the Care Provider “CP” would like to manually enter a diagnosis. The decision in decision block 953 is “YES” when the Care Provider “CP” has selected the selectable option. Otherwise, the decision in decision block 953 is “NO” when the Care Provider “CP” has not selected the selectable option and has instead selected one or more of the diagnoses from the list.

When the decision in decision block 953 is “NO,” in block 954, the system 100 identifies one or more Case Cards (“CCards”) associated with the one or more diagnoses selected in block 952 and instructs the expert user interface 122 to display the one or more CCards identified. A CCard is a collection of information related to a particular diagnosis. By way of a non-limiting example, a CCard may display the following information:

Diagnosis;

Explanation;

General advice;

Expectations;

Warnings;

When to call the Care Provider “CP;”

Follow up;

Medication—Recommendations; and

Investigations—Recommendations.

The system 100 may store one or more CCards for each diagnosis in the list. For example, the system 100 may have separate CCards for a single diagnosis based on levels of severity, age of the Care Recipient “CR,” etc.

In block 957, the Care Provider “CP” may mark the information of each of the one or more CCards (displayed by the expert user interface 122) as visible or hidden. Visible information will be viewable by the Care Giver “CG” in the non-expert user interface 120 when the Care Giver “CG” reviews the Expert Opinion in the method 500 (illustrated in FIG. 6A). Hidden information is not displayed by the non-expert user interface 120. Further, the information in the CCards may be editable by the Care Provider “CP” so that the Care Provider “CP” may tailor or customize the information presented to the Care Giver “CG” by the CCards. Edited CCards may be stored as templates in a private list in a manner substantially similar to that described below with respect to block 970.

Then, the system 100 advances to decision block 958.

When the decision in decision block 953 is “YES,” in block 955, the system 100 instructs the expert user interface 122 to display a blank CCard. The blank CCard includes data inputs (or fields) into which information may be entered by the Care Provider “CP.”

In block 956, the system 100 receives the information entered into the blank CCard by the Care Provider “CP” and instructs the expert user interface 122 to display a selectable option indicating the Care Provider “CP” would like to store the CCard (into which information was just entered in block 956) as a template.

In decision block 969, the system 100 determines whether the Care Provider “CP” has selected the selectable option displayed in block 956. The decision in decision block 969 is “YES” when the Care Provider “CP” has selected the selectable option. Otherwise, the decision in decision block 969 is “NO” when the Care Provider “CP” has not selected the selectable option.

When the decision in decision block 969 is “YES,” in block 970, the system 100 adds the CCard (into which information was entered in block 956) to a private CCard list associated with the Care Provider “CP” for access thereby. The CCard includes a diagnosis that was entered manually by the Care Provider “CP” in the blank CCard displayed in block 955. Whenever the system 100 receives an indication that the Care Provider “CP” has selected a diagnosis (in block 952) associated with a CCard in the private list, in block 954, the system 100 may instruct the expert user interface 122 to also display the CCard(s) associated with the diagnosis stored in the private list. The system 100 may also store any manually entered diagnoses for inclusion in the list displayed in block 951. Optionally, the system 100 may display a manually entered diagnosis to only the care provider who entered the diagnosis. Thus, each care provider may assemble a list of manually entered diagnoses viewable only by the care provider and a private list of CCards associated with the manually entered diagnoses.

After block 970, the system 100 advances to optional decision block 958.

When the decision in decision block 969 is “NO,” the system 100 advances to optional decision block 958.

Optionally, the Care Provider “CP” may indicate the Care Provider “CP” would like to issue one or more prescriptions. In optional decision block 958, the system 100 determines whether the Care Provider “CP” has indicated (by a command entered into the expert user interface 122) the Care Provider “CP” would like to issue one or more prescriptions. The decision in decision block 958 is “YES” when the Care Provider “CP” has indicated that the Care Provider “CP” would like to issue one or more prescriptions. Otherwise, the decision in decision block 958 is “NO.” In decision block 958, the expert user interface 122 may display a question asking the Care Provider “CP” whether the Care Provider “CP” would like to issue one or more prescriptions.

When the decision in decision block 958 is “YES,” in block 959, the system 100 configures the expert user interface 122 to receive prescription information that may be used to generate one or more electronic prescriptions (eprescriptions) that may be filled by a pharmacy. Then, the system 100 advances to optional decision block 960.

When the decision in decision block 958 is “NO,” the system 100 advances to optional decision block 960.

Optionally, the Care Provider “CP” may indicate the Care Provider “CP” would like to order or recommend one or more investigations. In optional decision block 960, the system 100 determines whether the Care Provider “CP” has indicated (by a command entered into the expert user interface 122) the Care Provider “CP” would like to recommend one or more investigations. When an investigation is recommended, the system 100 may help the Care Giver “CG” find the location whereat the investigation will be conducted. The system may also help locate where investigations can be performed or from which vendors an electronic order for an investigation can be placed. The system 100 may be configured to place such electronic orders. The system 100 may include geolocation capabilities that may be used to help choose vendors based on their proximity to the Care Giver “CG” and/or the Care Provider “CP.” Thus, wherever there is a service location, the system 100 may use geolocation as a basis for selection and providing selectable options. The decision in decision block 960 is “YES” when the Care Provider “CP” has indicated that the Care Provider “CP” would like to recommend one or more investigations. Otherwise, the decision in decision block 938 is “NO.” In decision block 960, the expert user interface 122 may display a question asking the Care Provider “CP” whether the Care Provider “CP” would like to recommend one or more investigations.

When the decision in decision block 960 is “YES,” in block 961, the system 100 configures the expert user interface 122 to receive recommendations for one or more investigations that may be used to conduct such investigations (einvestigations). Optionally, the system 100 may perform the method 1100 in block 961. Then, the system 100 advances to optional decision block 962.

When the decision in decision block 960 is “NO,” the system 100 advances to optional decision block 962.

Turning to FIG. 10B-2, the Care Provider “CP” may indicate that the Care Provider “CP” would like to add one or more Protocol Inserts to the Expert Opinion. In decision block 962, the system 100 determines whether the Care Provider “CP” has indicated (by a command entered into the expert user interface 122) the Care Provider “CP” would like to add one or more Protocol Inserts to the Expert Opinion. A Protocol Insert includes general information related to a particular condition or symptom that may be common to more than one diagnosis. For example, the system 100 may store a Protocol Insert for dehydration, a condition that is common to many illnesses. Protocol Inserts may be characterized as action driven protocols that focus on the treatment of generic issues like hydration, eating, temperature management, etc.

If the Care Provider “CP” believes the Care Giver “CG” should view the information included in the Protocol Insert for dehydration, the Care Provider “CP” may include it in the Expert Opinion. By way of a non-limiting example, the Protocol Inserts may store the same types of information that is stored by the CCards. In decision block 962, the expert user interface 122 may display a question asking the Care Provider “CP” whether the Care Provider “CP” would like to add one or more Protocol Inserts to the Expert Opinion.

The decision in decision block 962 is “YES” when the Care Provider “CP” has indicated that the Care Provider “CP” would like to add one or more Protocol Inserts to the Expert Opinion. Otherwise, the decision in decision block 962 is “NO.”

When the decision in decision block 962 is “YES,” in block 966, the system 100 instructs the expert user interface 122 to display a list of Protocol Inserts. The Protocol Inserts in the list are selectable by the Care Provider “CP.” Optionally, the list may include only Protocol Inserts associated with the diagnosis or information in one or more of the CCards added to the Expert Opinion. Alternatively, the list may include all Protocol Inserts stored by the system 100 (e.g., in a database).

In block 972, the system 100 receives an indication from the CP computing device 112 that the Care Provider “CP” has selected one or more Protocol Inserts, and advances to optional decision block 963.

When the decision in decision block 962 is “NO,” the system 100 advances to optional decision block 963.

Optionally, the Care Provider “CP” may indicate the Care Provider “CP” would like to recommend a referral to a different care provider (e.g., to a specialist). In optional decision block 963, the system 100 determines whether the Care Provider “CP” has indicated (by a command entered into the expert user interface 122) the Care Provider “CP” would like to recommend a referral to a different care provider (e.g., a specialist). In decision block 963, the expert user interface 122 may display a question asking the Care Provider “CP” whether the Care Provider “CP” would like to recommend a referral to a different care provider. The decision in decision block 963 is “YES” when the Care Provider “CP” has indicated that the Care Provider “CP” would like to recommend a referral to a different care provider. Otherwise, the decision in decision block 963 is “NO.”

When the decision in decision block 963 is “YES,” in block 964, the system 100 instructs the expert user interface 122 to display a free form text box into which the Care Provider “CP” may enter additional notes. The note(s) entered may be for the Care Giver “CG.” If any notes are entered by the Care Provider “CP,” in block 964, these notes are captured by the system 100. Next, in block 974, the system 100 performs the method 1300 illustrated in FIG. 14. Then, the system 100 advances to optional decision block 971.

When the decision in decision block 963 is “NO,” in block 965, the system 100 instructs the expert user interface 122 to display a free form text box into which the Care Provider “CP” may enter additional notes. The note(s) entered may be for the Care Giver “CG.” If any notes are entered by the Care Provider “CP,” in block 965, these notes are captured by the system 100. Then, the system 100 advances to optional decision block 971.

In optional decision block 971, the system 100 may determine whether the core system 114 stores any preprepared information (e.g., information provided by one or more third parties) related to the one or more diagnoses and/or medical protocols. The decision in optional decision block 971 is “YES” when the core system 114 stores information related to the one or more diagnoses and/or medical protocols. On the other hand, the decision in optional decision block 971 is “NO” when the core system 114 does not store information related to the one or more diagnoses and/or medical protocols.

When the decision in optional decision block 971 is “YES,” in block 973, the core system 114 sends the preprepared information to the CG computing device 110 for display thereby to the Care Giver “CG.” Then, the system 100 advances to block 967.

When the decision in optional decision block 971 is “NO,” the system 100 advances to block 967.

At this point, the Care Provider “CP” has finished entering the information for the Expert Opinion into the expert user interface 122. In block 967, the expert user interface 122 receives a command from the Care Provider “CP” to submit the information for the Expert Opinion. The expert user interface 122 submits the information for the Expert Opinion to the system 100 for processing in response to receiving a command from the Care Giver “CG” to submit the information for the Expert Opinion. Then, the method 950 terminates.

After the method 950 terminates, the Expert Opinion has been submitted to the system 100 for processing thereby. For example, the method 1400 (described below and illustrated in FIG. 15) may be performed by the core system 114 after the completion of the method 950.

### Method 1000: Request Additional Details

Turning to FIG. 11, the method 1000 configures the expert user interface 122 for display on the CP computing device 112 and receives input from the Care Provider “CP.”

In first block 1010, the system 100 configures the expert user interface 122 to receive one or more questions from the Care Provider “CP” to be displayed to the Care Giver “CG” by the non-expert user interface 120.

Then, in block 1020, the system 100 sends a notification to the CG computing device 112 indicating that a request for additional details has been received from the Care Provider “CP.”

Then, the method 1000 terminates.

### Method 1100: Suggest Investigation(s)/Test(s)

Turning to FIG. 12, the method 1100 configures the expert user interface 122 for display on the CP computing device 112 and receives input from the Care Provider “CP.”

In first block 1110, the system 100 configures the expert user interface 122 to receive information related to one or more investigations and/or tests suggested by the Care Provider “CP” to be displayed to the Care Giver “CG” by the non-expert user interface 120. For example, the Care Provider “CP” may wish to recommend a particular test. In the block 1110, the Care Provider “CP” may enter information in the expert user interface 122 explaining this recommendation and providing information related thereto.

Then, in block 1120, the system 100 sends a notification to the CG computing device 112 indicating that investigations/tests have been suggested by the Care Provider “CP.”

Then, the method 1100 terminates.

### Method 1200: Suggest Specialist Review

Turning to FIG. 13, the method 1200 configures the expert user interface 122 for display on the CP computing device 112 and receives input from the Care Provider “CP.”

In first block 1210, the system 100 configures the expert user interface 122 to receive information related to a consultation with a Specialist suggested by the Care Provider “CP” to be displayed to the Care Giver “CG” by the non-expert user interface 120. For example, the Care Provider “CP” may wish to suggested or recommend a consultation with a particular type of specialist, a consultation with a particular Specialist, and the like. In the block 1210, the Care Provider “CP” may enter information in the expert user interface 122 explaining this recommendation and providing information related thereto. For example, in block 1210, the Care Provider “CP” may enter a type of specialist from whom a consultation should be sought. By way of another example, in block 1210, the Care Provider “CP” may identify a particular specialist from whom a consultation should be sought.

Then, in block 1220, the system 100 sends a notification to the CG computing device 112 indicating that a consultation with a Specialist has been suggested by the Care Provider “CP.”

Then, the method 1200 terminates.

### Method 1300: Urgent Case—Call Care Giver “CG”

Turning to FIG. 14, the method 1300 is performed when the Care Provider “CP” has determined the Live Case is urgent. When this occurs, in block 1310, the Care Provider “CP” contacts the Care Giver “CG” directly. For example, the Care Provider “CP” may call the Care Giver “CG” on the telephone, send an urgent text message to the Care Giver “CG,” and the like.

Urgency may be depicted on a scale. For example, a scale of urgency from most urgent to least urgent may include the following:

emergencies,

urgent cases,

high acuity cases,

non-urgent routine cases, and

general queries.

Live cases may be tagged according to their urgency, which allows the system 100 to perform an intelligent triage and prioritization of Live Cases as well as to structure and modify the medical protocols displayed to the Care Provider “CP” for selection thereby.

The Care Provider “CP” then enters information into the expert user interface 122 that is captured by the system 100 and used to create an “urgent” case report. For example, the Care Provider “CP” may be presented with one or more fields into which telephone call notes (conversation notes), physician's notes, additional protocols, and/or follow up protocol details may be entered. The “urgent” case report may follow the same “process” as a regular or non-urgent case report.

For example, in next block 1320, the system 100 may instruct the expert user interface 122 to display a free form text box into which the Care Provider “CP” may enter a conversation note. If a conversation note is entered by the Care Provider “CP,” in block 1320, this note is captured by the system 100.

In next block 1320, the system 100 allows the Care Provider “CP” to insert one or more Protocol Inserts for display to the Care Giver “CG.” The protocol inserts will be displayed to the Care Giver “CG” (on the non-expert user interface 120) so the Care Giver “CG” can review the Protocol Inserts and if the Protocol Inserts include any instructions, perform any tasks included in the Protocol Inserts.

In block 1340, the system 100 instructs the expert user interface 122 to display a free form text box into which the Care Provider “CP” may enter additional notes. The notes may be for the Care Giver “CG.” If any notes are entered by the Care Provider “CP,” in block 1340, these notes are captured by the system 100.

At this point, the Care Provider “CP” has finished entering the information for the Expert Opinion into the expert user interface 122. In block 1345, the expert user interface 122 receives a command from the Care Provider “CP” to submit the information for the Expert Opinion. The expert user interface 122 submits the information for the Expert Opinion to the system 100 for processing in response to receiving a command from the Care Giver “CG” to submit the information for the Expert Opinion. Then, the method 950 terminates.

After the method 950 terminates, the Expert Opinion has been submitted to the system 100 for processing thereby. For example, the method

Then, the method 1300 terminates.

### Method 1400: Process Expert Opinion

After the method 900 terminates, the Expert Opinion has been submitted to the system 100 for processing thereby. At that point, the method 1400 illustrated in FIG. 15 may be performed by the core system 114.

In first block 1410, the core system 114 extracts data from the one or more medical protocols included in the Expert Opinion. The extracted information may include relevant medical history information that is important for triage (e.g., allergies, medication history, height, and weight). This extraction may be helpful because the health record often is too long for the Care Provider “CP” to read. Instead, the Care Provider “CP” typically only wants certain pieces of information to formulate a medical protocol. The extracted data is stored in the Live Case database (described below) and associated with the Care Recipient “CR.” Thus, the extracted data is added to the Health details associated with the Care Recipient “CR.”

In next block 1420, the core system 114 identifies additional information in the external data sources (not shown) relevant to the Expert Opinion and combines the Expert Opinion and the additional information (and/or links thereto) with the Expert Opinion to create an “Opinion Request Report.” Links to the external data sources may be stored in the knowledge base database (described below). The additional information (or the links thereto) is stored in the Live Case database (described below) and associated with the Expert Opinion.

Optionally, the method 1400 may translate the information of the Expert Opinion into an automatically generated plain-language version of the Expert Opinion to be included in the Opinion Request Report. Thus, the system 100 may be characterized as converting the expert-language Expert Opinion (provided by the Care Provider “CP”) into a lay or plain-language format using plain (non-expert) language for review by the Care Giver “CG.” The process of creating this plain language version is the reverse of the process used to convert the plain language description of the issue into the expert language included in the Intelligent Resident Note. In other words, the same data used to construct the Intelligent Resident Note is used to construct the plain language version of the Expert Opinion.

Then, in block 1430, the core system 114 sends a notification to the CG computing device 120 indicating that the Expert Opinion is ready for review by the Care Giver “CG.”

After the predetermined amount of time has elapsed, in decision block 1440, the system 100 determines whether the Opinion Request Report has been reviewed. The decision in decision block 750 is “YES” when the Opinion Request Report has not been reviewed within the predetermined amount of time. An amount of time that elapsed between the submission of the Expert Opinion and the review of the Expert Opinion may be displayed to the Care Provider “CP” in the expert user interface 122 to provide transparency related to the review time. Otherwise, the decision in decision block 1440 is “NO” when the Opinion Request Report has been reviewed within the predetermined amount of time.

When the decision in decision block 1440 is “NO,” the method 1400 terminates.

On the other hand, when the decision in decision block 1440 is “YES,” in block 1450, the system 100 sends a notification to the Care Provider “CP” via the expert user interface 122 so that the Care Provider “CP” may contact the Care Giver “CG.” Then, the method 1400 terminates.

Even if the predetermined amount of time has not elapsed, the amount of time that has elapsed may be displayed to the Care Provider “CP,” who may use this information (and the urgency or prioritization tagging) to escalate and/or manage triage that is asynchronous with the arrival of Live Cases. The escalation and/or management of triage may be conveyed back to the Care Giver “CG” via the non-expert user interface 120. Using the non-expert user interface 120, the Care Giver “CG” may escalate (and/or increase the urgency of) an Opinion Request if circumstances change and Live Case is becoming more or less urgent.

The Opinion Request Report may be displayed to the Care Giver “CG” in the method 500 illustrated in FIG. 6A.

The system 100 aids in the dialogue with an expert service provider to enhance their capability to render an expert opinion and provide them the tools to facilitate the protocol as well as be able to measure and assess adherence/compliance to the protocol. The system 100 may enhance the scheduling, prioritization, and delivery of care advice by the caregiver. By appropriately interrogating and tagging relevant information, the system 100 provides a brief yet intelligent and contextual (relevant medical history or past history) picture of the clinical event with clear capture and documentation of vital signs (captured manually or direct from an instrument), primary complaints and other relevant medical details. The system 100 may be configured to provide analytics and measurement capabilities, with easy to use scales that will chart across a journal the relevant health status and development of the child. The system 100 may manage by way of reminders and alerts various follow up or intervention care scenarios.

The system 100 may provide an electronic framework for the Care Giver “CG” to monitor, track, and document their Care Recipient's health on an ongoing basis to help the Care Giver “CG” provide proper and accurate information to the Care Provider “CP” during in office visit, instead of relying only on the memory of the Care Giver as was traditionally the case.

All relevant health information (both a single event or events occurring over a period of time) for a given Care Recipient “CR” may be compared to published guidelines (quantitative and qualitative) and the system 100 may highlight deviations and provide informative input to the Care Giver “CG” to make the relevant decision for action.

The system 100 may be configured to interrogate the Care Giver “CG” for information about the condition or problem being rendered for assessment, to provide richer and more complete linkage with relevant information, to use Live Case data to drive external and internal product and service and information linkage, to build records and knowledge of trends and information relevant to future issues and visits by assimilating and linking live cases.

### Data Tagging

Information entered into the system 100 by the Care Giver “CG” via the non-expert user interface 122 and information entered into the system 100 by the Care Provider “CP” via the expert user interface 120 is tagged with known medical dictionary terminology and stored. A medical definition dictionary may be used to identify key words for tagging. Terms may be segmented based on their relevance to the screening process.

Other data, such as that generated by the tracking functions (described above) is also tagged with known medical dictionary terminology and stored. In particular embodiments, all information collected and requested during and after a Live Case is tagged with known medical dictionary terminology. Additionally, information generated by the system during and after the Live Case may also be tagged with known medical dictionary terminology.

These tags may be used to filter, sort, and/or prioritize the information. In particular, each piece of information may be tagged with a category selected from a set of predefined categories (e.g., a Diagnosis category, an Investigation category, and a Treatment category).

The tags may be used to implement searching, sorting, and delivery of information. Thus, the tags may be used to link products, services, and/or information relevant to the Care Giver “CG,” Care Provider “CP,” and/or Care Recipient “CR.” Machine and intelligent learning techniques applied to search activities associated with the Care Giver “CG,” Care Provider “CP,” and/or Care Recipient “CR” may be used to modify delivery of products, services, and/or information.

For example, tags may be used to link relevant information in the Live Case, health profile, health album, and/or geolocation to medical terms categorized as being related to diagnosis (“Dx”), investigation (“Ix”), and/or treatment (“Rx”). By categorizing the relevant information in this manner, the system may provide intuitive and higher yield information products and services capabilities.

When the Care Giver “CG” is searching for medical information, typically, the Care Giver “CG” is asking for the following information with respect to the issue and/or the Expert Opinion:
1. Questions related to Diagnosis: what is it that the Care Recipient “CR” has? Care Giver “CG” is requesting information, clarity, differential, prognosis, etc.
2. Questions related to Investigation: how do I make sure this is what the Care Recipient “CR” has? Care Giver “CG” is requesting details on tests and further exploration of the pathway to understand the prognosis of the condition.
3. Questions related to Treatment: how can the Care Giver “CG” manage the problem and what are the Care Giver's “CG” options? Is this the best option for treatment? Care Giver “CG” is seeking optimal and alternative options for management.

The system 100 links the medically tagged information from a Live Case to past event history and combines the medically tagged information with personal relevant health record (that is the Health Profile associated with the Care Recipient “CR”) including biometric data to analyze likely and probable issues given this data. This enables a more structured search for the options regards investigation and management of the condition.

The system 100 utilizes the basic data and conclusion sets that define the relevance of data presented to the Care Giver “CG:”
1. Biodata of Care Recipient “CR”—age and gender and relevant allergies, pharmaceutical history, and the like;
2. Case event log of Care Recipient “CR”—frequency, timing, and response to treatment of the collection of Diagnoses the Care Recipient “CR” has received and the investigations including result of collated with treatment and response(s) thereto; and
3. Differential diagnosis of Care Recipient “CR”—an intelligent summation of potential issues given the information from items 1 and 1 above.

Using this data and synthesis of information with derived conclusions of probable issues and questions, the system 100 searches appropriate sources of information that will improve the likelihood of locating relevant information.

The system 100 may then categorize this information as being related to diagnosis (“Dx”), investigation (“Ix”), and/or treatment (“Rx”) so that the Care Giver “CG” can better synthesize and store the information. These categories may overlap and depending upon the implementation details terms may appear in more than one category. The system 100 is configured to receive an acknowledgement of relevant information on a scale (indicating how relevant the information located is) to better refine a search. Each piece of information is associated with specific terminology that once tagged can be utilized in an advanced search process.

### Core System 114

Data stored by the core system 114 may be arranged in logical collections (referred to as databases) of related information. While the information is described as being stored in databases those of ordinary skill in the art appreciate that the data may be stored using any data structures known in the art including data files, linked lists, relational database tables, and the like.

The data stored by the core system 114 may include a user database that stores information related to the Care Giver “CG” and details related to the Care Recipient “CR” and a link to one or more preferred care provider in the Care Provider Network “CG NETWORK.” The user database may be populated by the Care Giver “CG” when the Care Giver “CG” registers with the system 100. The system 100 is not limited to use with a particular registration process. During the registration process, the Care Giver “CG” may select a language in which the non-expert user interface 120 is presented to the Care Giver “CG.”

The data stored by the core system 114 may include a patient database that stores health history, health profile, prescription history, growth history, development tracking/history, vaccine information, dental/vision/earring history, and condition/emotion tracking (scale). At least a portion of the patient database may be populated by the Care Giver “CG” using the method 650 illustrated in FIG. 7B. The patient database may also be populated by information provided by the Care Giver “CG” in Opinion Requests and the Care Provider “CP” in the Expert Opinion. The data stored in the patient database may be used to generate Health and/or Developmental Monitoring graphical displays, such as those depicted in FIGS. 7C and 7E.

The data stored by the core system 114 may include a Care Provider database that stores information related to the care providers, such as general information (e.g., name), specialty, availability, schedule, and contact information. The Care Provider database may be populated by the Care Provider “CP” when the Care Provider “CP” registers with the system 100. The system 100 is not limited to use with the registration process. During the registration process, the Care Provider “CP” may select a language in which the expert user interface 122 is presented to the Care Provider “CP.” The expert user interface 122 may be presented to the Care Provider “CP” in a different language than the one used to present the non-expert user interface 120 to the Care Giver “CG.” By way of a non-limiting example, conventional translation software may be used to present the non-expert user interface 120 and expert user interface 122 in different languages.

The data stored by the core system 114 may include a Live Case database that stores information related to Live Cases, such as Primary Complaint, symptom(s), prescriptions, intensity (scale), urgency, additional information, and status. The Live Case database is populated at least in part by information provided by the Care Giver “CG” in Opinion Requests and the Care Provider “CP” in the Expert Opinion.

The Live Case database may be linked to a data file database configured to store any data files uploaded as part of the Opinion Request submission. The data file database stores tags associated with each of the data files.

The user database, patient database, Care Provider database, Live Case database, and data file database all store information related to specific Care Givers, Care Recipients, and/or Care Providers.

The data stored by the core system 114 may also include information related to Medical Protocols. For example, a protocol database may be used to store information related to conforming protocols, such as protocol details, a link to what PC, Options, Action(s), Treatment, Demographic/Gender, and dosage/applicability.

The data stored by the core system 114 may also include a PC database that stores information related to Primary Complaints and/or symptoms. The PC database may store details related to the forgoing, ranking information used for triage, chronic condition implications, related questions, and sub-primary complaints.

The data stored by the core system 114 may also include a prescription database that stores information related to prescriptions. This information may be supplied by conventional sources of such information.

The data stored by the core system 114 may also include a knowledge base database that stores links to external data sources that provide health information, prescription information, medical protocol information, device information, and/or management information.

### Additional Aspects of the System 100

The non-expert user interface 120 may be configured by the core system 114 to display options to the Care Giver “CG” that express urgency of the Opinion Request. The urgency may be tagged by the PC and Live Case details for definition of acuity and representation of urgency to the Care Provider “CP.”

The Care Provider “CP” may independently manage Opinion Requests assigned to the Care Provider to describe triage level urgency and prioritization tools for flagging and tagging cases to better manage the queue of incoming requests.

The non-expert user interface 120 may be configured by the core system 114 to display amounts of time taken by each Care Provider “CP” and to provide the option to escalate or reroute the Live Case to a more available Care Provider “CP.” The non-expert user interface 120 may be configured by the core system 114 to display the location and specialist description of the Care Provider “CP” to provide a better definition of choices available to the Care Giver “CG” when rerouting a Live Case.

The non-expert user interface 120 may be configured by the core system 114 to provide a way for the Care Provider “CP” to manage availability schedule and expectations for turnaround.

The expert user interface 122 may be configured by the core system 114 to provide the Care Provider “CP” with a real time status override. Using this feature, the Care Provider “CP” logs into the system 100 and notifies the system 100 that the Care Provider is “live” on the system and available. The non-expert user interface 120 may be configured by the core system 114 to display to Care Giver “CG” that the Care Provider is available on the system 100.

The system 100 may include geolocation capabilities that may be used to optimize data algorithms. The geolocation capabilities may provide location information that the system 100 may use to help the Care Giver “CG” select a Care Provider “CP.” For example, the system 100 may suggest a Care Provider “CP” to the Care Giver “CG” based on location information provided by the geolocation capabilities.

The system 100 (see FIG. 1) may be used to measure or evaluate use of standard protocols by care providers. For example, the system 100 may be used to determine how frequently a particular care provider edits a standard medical protocol and/or a management protocol. In other words, a conformance metric may be determined as a function of a number of the medical protocols edited by the care provider. After the method 900 has been performed a number of times by the Care Provider “CP,” the system 100 has collected a number of management protocols identified by the Care Provider “CP” each associated with a diagnosis and health information associated with the Care Recipient “CR.” The system 100 may also store a standard Management Protocol associated with each diagnosis. The management protocols selected by the Care Provider “CP” and sent to care givers may be compared to the standard protocols and a conformance metric determined as a function of a number of the medical protocols that vary from the standard management protocol.

The system 100 (see FIG. 1) may be used to archive medical images and other data for searching purposes. For example, after a substantial number of Live Cases have been completed (e.g., submitted, diagnosed, and any actions in the management protocols performed), data stored by the core system 114 may be queried. This data includes health information associated with a plurality of care recipients from a plurality of care givers. The health information may include a plurality of images each associated with at least one of the plurality of care recipients, a primary complaint, demographic variable values associated with the at least one of the plurality of care recipients, and responses to questions provided by one or more of the plurality of care givers. As discussed above with respect to method 220 illustrated in FIGS. 3A-1 and 3A-2, these questions are generated based at least in part on the primary complaint associated with the image. In response to a request for images associated with at least one search criteria, the system 100 may return any images satisfying the search criteria. By way of a non-limiting example, the search criteria may identify a particular primary complaint, a particular value of a selected one of the demographic variables, and/or a particular response to a selected question provided by one or more of the plurality of care givers. Any images identified as satisfying the search criteria may be forwarded to the computing device that requested the search.

The system 100 (see FIG. 1) may be used to broadcast messages (e.g., alerts) to care givers, care recipients, and/or care providers. As explained above, the system 100 stores medical data associated with a plurality of care recipients. A party wishing to broadcast information to care givers, care recipients, and/or care providers satisfying broadcast criteria may provide such criteria to the system. The system may then search the medical data to identify care givers, care recipients, and/or care providers that satisfy the broadcast criteria. Then, the system 100 may send a message to each of the care givers, care recipients, and/or care providers identified. Unlike conventional systems configured to send broadcast messages to all patients, the system 100 may send targeted broadcast messages. For example, the system 100 may send messages the care givers of all care recipients under the age of five who had ear infections treated with a particular drug in the past two years. By way of another example, the system 100 may send messages to all care providers who prescribed a particular drug to children with a particular diagnosis.

As explained above, the questions presented during the tailored health interrogation process 245 illustrated in FIG. 3A-1 may be arranged in a data structure, such as a tree. Further, the method 1000 illustrated in FIG. 11 may be used by the Care Provider “CP” to ask additional questions after receiving the Live Case. If the Care Provider “CP” repeatedly asks the same question when presented with the same Primary Complaint, the question may be added to the data structure associated with the Primary Complaint for the Care Provider “CP” or alternatively, for all or a portion of the care providers. For example, if the Care Provider “CP” asks the same question more than a threshold number of times with respect to a particular Primary Complaint, the question may be added to the data structure storing the questions for that Primary Complaint. For example, the system 100 may capture key words entered by the Care Provider “CP” in a follow up question associated with a Primary Complaint. If these key words are captured more than a threshold number of times with respect to the same Primary Complaint over a predetermined period of time, the system 100 concludes the question is relevant to the Primary Complaint and adds it to the data structure storing the questions for that Primary Complaint. Optionally, the question and/or key words may be reviewed by a person (e.g., an expert) before it is added to the data structure.

### Computing Environment

FIG. 16 is a diagram of hardware and an operating environment in conjunction with which implementations of the CG computing device 110, the computing device 111, the CP computing device 112, the computing device 113, the core system 114, and the network 116 may be practiced. The description of FIG. 16 is intended to provide a brief, general description of suitable computer hardware and a suitable computing environment in which implementations may be practiced. Although not required, implementations are described in the general context of computer-executable instructions, such as program modules, being executed by a computer, such as a personal computer. Generally, program modules include routines, programs, objects, components, data structures, etc., that perform particular tasks or implement particular abstract data types.

Moreover, those skilled in the art will appreciate that implementations may be practiced with other computer system configurations, including hand-held devices, multiprocessor systems, microprocessor-based or programmable consumer electronics, network personal computers, minicomputers, mainframe computers, and the like. Implementations may also be practiced in distributed computing environments where tasks are performed by remote processing devices that are linked through a communications network. In a distributed computing environment, program modules may be located in both local and remote memory storage devices.

The exemplary hardware and operating environment of FIG. 16 includes a general-purpose computing device in the form of a computing device 12. Each of the CG computing device 110, the CP computing device 112, and the core system 114 may be implemented in accordance with the computing device 12. The computing device 12 includes the system memory 22. The system memory 22 may store instructions executable by the processing unit 21.

The computing device 12 also includes a processing unit 21, and a system bus 23 that operatively couples various system components, including the system memory 22, to the processing unit 21. There may be only one or there may be more than one processing unit 21, such that the processor of computing device 12 comprises a single central-processing unit (CPU), or a plurality of processing units, commonly referred to as a parallel processing environment. The computing device 12 may be a conventional computer, a distributed computer, or any other type of computer.

The system bus 23 may be any of several types of bus structures including a memory bus or memory controller, a peripheral bus, and a local bus using any of a variety of bus architectures. The system memory may also be referred to as simply the memory, and includes read only memory (ROM) 24 and random access memory (RAM) 25. A basic input/output system (BIOS) 26, containing the basic routines that help to transfer information between elements within the computing device 12, such as during start-up, is stored in ROM 24. The computing device 12 further includes a hard disk drive 27 for reading from and writing to a hard disk, not shown, a magnetic disk drive 28 for reading from or writing to a removable magnetic disk 29, and an optical disk drive 30 for reading from or writing to a removable optical disk 31 such as a CD ROM, DVD, or other optical media.

The hard disk drive 27, magnetic disk drive 28, and optical disk drive 30 are connected to the system bus 23 by a hard disk drive interface 32, a magnetic disk drive interface 33, and an optical disk drive interface 34, respectively. The drives and their associated computer-readable media provide nonvolatile storage of computer-readable instructions, data structures, program modules, and other data for the computing device 12. It should be appreciated by those skilled in the art that any type of computer-readable media which can store data that is accessible by a computer, such as magnetic cassettes, flash memory cards, USB drives, digital video disks, Bernoulli cartridges, random access memories (RAMs), read only memories (ROMs), and the like, may be used in the exemplary operating environment. As is apparent to those of ordinary skill in the art, the hard disk drive 27 and other forms of computer-readable media (e.g., the removable magnetic disk 29, the removable optical disk 31, flash memory cards, USB drives, and the like) accessible by the processing unit 21 may be considered components of the system memory 22.

A number of program modules may be stored on the hard disk drive 27, magnetic disk 29, optical disk 31, ROM 24, or RAM 25, including an operating system 35, one or more application programs 36, other program modules 37, and program data 38. A user may enter commands and information into the computing device 12 through input devices such as a keyboard 40 and pointing device 42. Other input devices (not shown) may include a microphone, joystick, game pad, satellite dish, scanner, or the like. These and other input devices are often connected to the processing unit 21 through a serial port interface 46 that is coupled to the system bus 23, but may be connected by other interfaces, such as a parallel port, game port, or a universal serial bus (USB) and non-wired interfaces like WiFi, Bluetooth, and infrared. A monitor 47 or other type of display device is also connected to the system bus 23 via an interface, such as a video adapter 48. In addition to the monitor, computers typically include other peripheral output devices (not shown), such as speakers and printers.

The computing device 12 may operate in a networked environment using logical connections to one or more remote computers, such as remote computer 49. These logical connections are achieved by a communication device coupled to or a part of the computing device 12 (as the local computer). Implementations are not limited to a particular type of communications device. The remote computer 49 may be another computer, a server, a router, a network PC, a client, a memory storage device, a peer device or other common network node, and typically includes many or all of the elements described above relative to the computing device 12. The remote computer 49 may be connected to a memory storage device 50. The logical connections depicted in FIG. 8 include a local-area network (LAN) 51 and a wide-area network (WAN) 52. Such networking environments are commonplace in offices, enterprise-wide computer networks, intranets and the Internet. The network 10 may include any of the aforementioned networking environments.

When used in a LAN-networking environment, the computing device 12 is connected to the local area network 51 through a network interface or adapter 53, which is one type of communications device. When used in a WAN-networking environment, the computing device 12 typically includes a modem 54, a type of communications device, or any other type of communications device for establishing communications over the wide area network 52, such as the Internet. The modem 54, which may be internal or external, is connected to the system bus 23 via the serial port interface 46. In a networked environment, program modules depicted relative to the personal computing device 12, or portions thereof, may be stored in the remote computer 49 and/or the remote memory storage device 50. It is appreciated that the network connections shown are exemplary and other means of and communications devices for establishing a communications link between the computers may be used.

The computing device 12 and related components have been presented herein by way of particular example and also by abstraction in order to facilitate a high-level view of the concepts disclosed. The actual technical design and implementation may vary based on particular implementation while maintaining the overall nature of the concepts disclosed.

The system memory 22 may store instructions executable by the processing unit 21. The instructions may implement at least a portion of one or more of the methods 200, 220, 300, 330, 400, 500, 550, 600, 650, 700, 800, 900, 950, 1000, 1100, 1200, 1300, and 1400. The system memory 22 may store the databases described above as being stored by the core system 114. Such instructions may be stored on one or more non-transitory computer or processor readable media.

### Mobile Communication Device

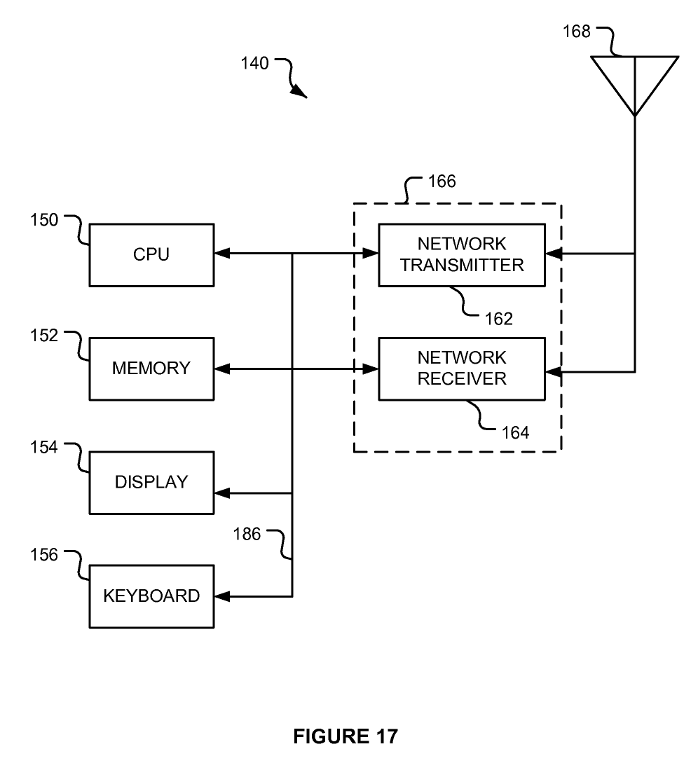

One or more of the CG computing device 110, the computing device 111, the CP computing device 112, and the computing device 113 may be implemented as mobile communication devices. FIG. 17 is a functional block diagram illustrating a mobile communication device that may be used to implement the CG computing device 110, the computing device 111, the CP computing device 112, and/or the computing device 113. The mobile communication device 140 includes a central processing unit (CPU) 150. Those skilled in the art will appreciate that the CPU 150 may be implemented as a conventional microprocessor, application specific integrated circuit (ASIC), digital signal processor (DSP), programmable gate array (PGA), or the like. The mobile communication device 140 is not limited by the specific form of the CPU 150.

The mobile communication device 140 also contains a memory 152. The memory 152 may store instructions and data to control operation of the CPU 150. The memory 152 may include random access memory, ready-only memory, programmable memory, flash memory, and the like. The mobile communication device 140 is not limited by any specific form of hardware used to implement the memory 152. The memory 152 may also be integrally formed in whole or in part with the CPU 150.

The mobile communication device 140 also includes conventional components, such as a display 154 (operable to display the expert user interface 122 and/or the non-expert user interface 120) and keypad or keyboard 156. These are conventional components that operate in a known manner and need not be described in greater detail. Other conventional components found in wireless communication devices, such as a USB interface, Bluetooth interface, camera/video device, infrared device, and the like, may also be included in the mobile communication device 140. For the sake of clarity, these conventional elements are not illustrated in the functional block diagram of FIG. 17.

The mobile communication device 140 also includes a network transmitter 162 such as may be used by the mobile communication device 140 for normal network wireless communication with a base station (not shown). FIG. 17 also illustrates a network receiver 164 that operates in conjunction with the network transmitter 162 to communicate with the base station (not shown). In a typical embodiment, the network transmitter 162 and network receiver 164 are implemented as a network transceiver 166. The network transceiver 166 is connected to an antenna 168. Operation of the network transceiver 166 and the antenna 168 for communication with a wireless network (not shown) is well-known in the art and need not be described in greater detail herein.

The mobile communication device 140 may also include a conventional geolocation module (not shown) operable to determine the current location of the mobile communication device 140.

The various components illustrated in FIG. 17 are coupled together by a bus system 186. The bus system 186 may include an address bus, data bus, power bus, control bus, and the like. For the sake of convenience, the various busses in FIG. 17 are illustrated as the bus system 186.

The memory 152 may store instructions executable by the CPU 150. The instructions may implement portions of one or more of the methods 200, 220, 300, 330, 400, 500, 550, 600, 650, 700, 800, 900, 950, 1000, 1100, 1200, 1300, and 1400. Such instructions may be stored on one or more non-transitory computer or processor readable media.

### Alternate Embodiments

The system 100 has been described as including the core system 114 that generates both the non-expert user interface 120 (displayed to the Care Giver “CG” by the CG computing device 110) and the expert user interface 122 (displayed to the Care Provider “CP” by the CP computing device 112). However, through application of ordinary skill in the art to the present teachings, embodiments of the methods described above may be implemented using short message service (“SMS”) technology and/or interactive voice response (“IVR”) technology. Therefore, such embodiments are within the scope of the present teachings

In such embodiments, the data requested and captured by the non-expert user interface 120 and/or the expert user interface 122 may be requested and captured using SMS and/or IVR technologies. Thus, instead of communicating via the CG computing device 110, the Care Giver “CG” and the system 100 may communicate via a conventional telephone, pager, device configured to implement text messaging, and the like. Similarly, instead of communicating via the CP computing device 112, the Care Provider “CP” and the system 100 may communicate via a conventional telephone, pager, device configured to implement text messaging, and the like.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

The invention claimed is:

1. A computer-implemented method for use with a care recipient experiencing a medical issue, the method comprising:
receiving an identification of the care recipient from a computing device operated by a care giver;
receiving identifications of one or more of primary complaints from the computing device operated by the care giver;
selecting a plurality of data collection questions based at least in part on the one or more primary complaints, the plurality of data collection questions being organized in a structure that determines at least in part an order in which responses are to be provided to the plurality of data collection questions, the order comprising a previous question to be responded to before a selected one of a plurality of subsequent questions, the selected one of the plurality of subsequent questions being selected at least in part based on a response to the previous question, the plurality of data collection questions and structure being configured to help identify a medical problem associated with the medical issue, but not to diagnosis the medical problem, the plurality of data collection questions being in non-expert language;
sending the plurality of data collection questions to the computing device operated by the care giver for display to the care giver in accordance with the order;
receiving a response to each of the plurality of data collection questions from the computing device operated by the care giver; and
sending one or more instructions to the computing device operated by the care giver related to treating the medical problem.

2. The method of claim 1, wherein the plurality of data collection questions and structure are further configured to help identify the severity of the medical problem, but not to diagnosis the severity of the medical problem.

3. The method of claim 1 for use with a health profile comprising health information related to the care recipient, the method further comprising:
formulating a list of selectable primary complaints based at least in part on the health information related to the care recipient for display to the care giver by the computing device operated by the care giver, wherein receiving the identifications of one or more of primary complaints from the computing device operated by the care giver comprises receiving a selection of the one or more of the selectable primary complaints from the computing device operated by the care giver.

4. The method of claim 1, further comprising before sending the one or more instructions to the computing device operated by the care giver:
formulating a non-expert language description of the medical issue;
sending the non-expert language description of the medical issue to the computing device operated by the care giver; and
receiving an indication that the care giver has accepted the non-expert language description of the medical issue.

5. The method of claim 1, further comprising:
identifying preprepared information associated with at least one of the one or more of primary complaints and the responses to the plurality of data collection questions; and
providing the preprepared information to the computing device operated by the care giver for display thereby.

6. The method of claim 1, wherein the one or more instructions comprise at least one action to be performed by the care giver, and the method further comprises:
receiving an indication from the computing device operated by the care giver that the at least one action has been performed.

7. The method of claim 1, wherein the one or more instructions comprise at least one action to be performed by the care giver, and the method further comprises:
sending one or more alerts to the computing device operated by the care giver prompting the care giver to perform the at least one action.

8. The method of claim 1, further comprising:
formulating an expert-language description of the medical issue after receiving the responses to the plurality of data collection questions from the computing device operated by the care giver;
sending the expert-language description of the medical issue to a computing device operated by a selected care provider; and
receiving the one or more instructions related to treating the medical problem from the computing device operated by the selected care provider.

9. The method of claim 1, further comprising:
receiving a first language selection, the plurality of data collection questions being formulated in accordance with the first language selection;
receiving a second language selection, the second language selection being different from the first language selection;
formulating an expert-language description of the medical issue in accordance with the second language selection after receiving the responses to the plurality of data collection questions from the computing device operated by the care giver;
sending the expert-language description of the medical issue to a computing device operated by a selected care provider; and
receiving the one or more instructions related to treating the medical problem from the computing device operated by the selected care provider, the one or more instructions sent to the computing device operated by the care giver being constructed in accordance with the first language selection.

10. The method of claim 1, further comprising:
formulating an expert-language description of the medical issue after receiving the responses to the plurality of data collection questions from the computing device operated by the care giver;
sending the expert-language description of the medical issue to a computing device operated by a first care provider;
receiving an identification of a second care provider from the computing device operated by the first care provider;
sending the expert-language description of the medical issue to a computing device operated by the second care provider; and
receiving the one or more instructions related to treating the medical problem from the computing device operated by the second care provider.

11. The method of claim 1, wherein the care giver is a member of a care giver network comprising a plurality of care givers, and the method further comprises:
sending the one or more instructions to computing devices operated by the members of the care giver network.

12. The method of claim 1, further comprising:
receiving an instruction from the computing device operated by the care giver that at least a portion of the one or more instructions are to be assigned to an alternate care giver; and
sending the portion of the one or more instructions to a computing device operated by the alternate care giver.

13. The method of claim 1, further comprising:
instructing the computing device operated by the care giver to display a list of care providers; and
receiving an identification of the selected care provider from the computing device operated by the care giver.

14. The method of claim 13, wherein the list of care providers was provided by the care giver.

15. The method of claim 13, further comprising:
selecting the care providers included in the list based on the geographic location of the computing device operated by the care giver.

16. The method of claim 1 further comprising:
after receiving the identifications of one or more primary complaints from the computing device operated by the care giver, sending a request requesting at least one of vital sign information associated with the care recipient and data related to observations of the care recipient to the computing device operated by the care giver.

17. The method of claim 1, wherein the structure is a tree.

18. The method of claim 1 for use with a health profile comprising health information related to the care recipient, wherein the plurality of questions is selected based at least in part on the health information related to the care recipient.

19. The method of claim 1 for use with a health profile comprising health information related to the care recipient, the health information comprising at least one of an age of the care recipient, a gender of the care recipient, and a geographic location of the care recipient, wherein the list of selectable primary complaints is formulated based at least in part on the at least one of the age of the care recipient, the gender of the care recipient, and the geographic location of the care recipient.

20. The method of claim 1 for use with a health profile comprising health information related to the care recipient, the health information identifying at least one chronic condition, wherein the list of selectable primary complaints is formulated based at least in part on the at least one chronic condition identified in the health profile.

21. A computer-implemented method comprising:
formulating an expert-language representation of health related information associated with a care recipient experiencing a medical issue, the health related information having been provided by a computing device operated by a care giver, at least a portion of the health related information having been provided in non-expert language;
sending the expert-language representation to a computing device operated by a care provider;
receiving a diagnosis;
identifying a medical protocol based on the diagnosis;
sending the medical protocol to the computing device operated by the care provider for display thereby;
receiving an acceptance of the medical protocol from the computing device operated by the care provider; and
sending a management protocol comprising at least a portion of the medical protocol to the computing device operated by the care giver for display thereby.

22. The method of claim 21, further comprising before the acceptance of the medical protocol is received from the computing device operated by the care provider:
instructing the computing device operated by the care provider to display an editable version of the medical protocol to the care provider; and
receiving edits to the medical protocol from the computing device operated by the care provider.

23. The method of claim 21, wherein the health related information provided by the computing device operated by the care giver comprises responses to questions provided by the care giver and health information stored in a health profile associated with the care recipient.

24. The method of claim 21, further comprising:
sending questions to the computing device operated by the care giver;
receiving responses to the questions from the computing device operated by the care giver, the information provided by the computing device operated by the care giver comprising the responses;
identifying at least one additional medical protocol based on one or more of the responses;
receiving an acceptance of the at least one additional medical protocol from the computing device operated by the care provider; and
instructing the computing device operated by the care giver to display at least one additional management protocol comprising at least a portion of the at least one additional medical protocol.

25. The method of claim 21, wherein the management protocol comprises at least one action to be performed by the care giver, and the method further comprises:
sending an indication to the computing device operated by the care provider when the at least one action has been performed.

26. The method of claim 21, wherein the management protocol comprises at least one action to be performed by the care giver, and the method further comprises:
in response to a request from the computing device operated by the care provider, instructing the computing device operated by the care provider to display an indication of whether the at least one action has been performed.

27. The method of claim 21, further comprising:
searching a medical history associated with the care recipient for past events and data related to at least one of the primary complaint and the information provided by the computing device operated by the care giver; and
instructing the computing device operated by the care provider to display to the care provider any of the past events and data located by the search.

28. The method of claim 27, further comprising:
instructing the computing device operated by the care provider to display a link to the medical history associated with the care recipient.

29. The method of claim 21, wherein the identified medical protocol was provided by the care provider.

30. The method of claim 21 for use with information supplied by a third party comprising preprepared information related to a plurality of diagnoses, the method further comprising:
identifying a portion of the information supplied by the third party comprising preprepared information related to the diagnosis; and
providing the identified portion of the information supplied by the third party to the computing device operated by the care giver for display thereby.

31. The method of claim 21, further comprising:
identifying preprepared information associated with at least one of the primary complaint and the information provided by the computing device operated by the care giver; and
providing the preprepared information to the computing device operated by the care giver for display thereby.

32. The method of claim 21 further comprising before receiving the diagnosis:
receiving a request from the computing device operated by the care provider for a response from the care giver;
instructing the computing device operated by the care giver to display the request;
receiving a response to the request from the computing device operated by the care giver; and
sending the response to the request to the computing device operated by the care provider.

33. The method of claim 21 further comprising before receiving the diagnosis and the indication of severity:
receiving a request from the computing device operated by the care provider for at least one of a data file and a data stream;
instructing the computing device operated by the care giver to display the request;
receiving at least one of a data file and a data stream in response to the request from the computing device operated by the care giver; and
sending the received at least one of the data file and the data stream to the computing device operated by the care provider.

34. The method of claim 33, wherein the received at least one of the data file and the data stream comprises an image file, a video file, an audio file, or a rich media file.

35. The method of claim 33, further comprising before sending the received at least one of the data file and the data stream to the computing device operated by the care provider:
sending a request for tag information for the requested at least one of the data file and the data stream to the computing device operated by the care giver;
receiving the tag information from the computing device operated by the care giver;
comparing the tag information to the request for the at least one of the data file and the data stream to identify a mismatch; and
if a mismatch is identified, instructing the computing device operated by the care giver to display a request for another at least one of a data file and a data stream.

36. A computer-implemented method comprising:
collecting health information associated with a plurality of care recipients from a plurality of care givers, the health information comprising a plurality of images each associated with at least one of the plurality of care recipients, a primary complaint, demographic variable values associated with the at least one of the plurality of care recipients, and responses to questions provided by one or more of the plurality of care givers, the questions having been generated based at least in part on the primary complaint associated with the image;
receiving a request for images associated with at least one search criteria from a requesting computing device, the at least one search criteria identifying at least one of a particular primary complaint, a particular value of a selected one of the demographic variables, and a particular response to a selected question provided by one or more of the plurality of care givers;
identifying ones of the plurality of images satisfying the at least one search criteria; and
forwarding the identified ones of the plurality of images to the requesting computing device.

37. A computer-implemented method comprising:
instructing a computing device operated by a care provider to display a plurality of primary complaints each describing a medical issue of a care recipient;
receiving a diagnosis and an indication of severity for each of the plurality of primary complaints from the computing device operated by the care provider;
identifying a medical protocol for each of the plurality of primary complaints based on the diagnosis and the indication of severity received for the primary complaint;
instructing the computing device operated by the care provider to display to the care provider an editable version of the medical protocol for each of the plurality of primary complaints;
receiving edits to at least a portion of the medical protocols from the computing device operated by the care provider; and
determining a compliance metric for the care provider as a function of a number of the medical protocols edited by the care provider.

38. A computer-implemented method comprising:
storing medical data associated with a plurality of care recipients, a different portion of the medical data being associated with each of the plurality of care recipients, each different portion of the medical data comprising a primary complaint and responses to questions provided by at least one of the plurality of care givers and a diagnosis and management protocol provided by a care provider;
identifying broadcast criteria;
searching the medical data using the broadcast criteria to identify ones of the plurality of care recipients associated with portions of the medical data that satisfy the broadcast criteria; and
sending a message to each of the identified ones of the plurality of care recipients.

39. The method of claim 38 wherein each different portion of the medical data further comprises at least one of vital sign information associated with the care recipient and data related to observations of the care recipient provided by the at least one of the plurality of care givers.

40. The method of claim 38 wherein each different portion of the medical data further comprises at least one data file provided by the at least one of the plurality of care givers.

41. The method of claim 38 wherein the broadcast criteria identifies a particular primary complaint.

42. The method of claim 38 wherein the broadcast criteria identifies a particular response to a particular question.

43. A computer-implemented method comprising:
for each of a plurality of care recipients, receiving an identification of a primary complaint, the primary complaint being substantially identical for each of a plurality of care recipients;
for each of the plurality of care recipients, sending a set of questions associated with the primary complaint to a computing device operated by a care giver;
for each of the plurality of care recipients, receiving responses to the set of questions;
for each of the plurality of care recipients, sending the responses to a computing device operated by a care provider;
for each of at least a portion the plurality of care recipients, receiving a new question from the computing device operated by the care provider; and
adding the new question to the set of questions associated with the primary complaint.

44. A system for use with a care giver computing device and a care provider computing device, the system comprising:
(a) one or more data storage devices storing a plurality of data collection questions associated with a primary complaint, the plurality of data collection questions being in non-expert language; and
(b) a core computing system configured to (i) receive an identification of the primary complaint from the care giver computing device, (ii) send selected ones of the plurality of data collection questions to the care giver computing device, the selected ones comprising subsequent data collection questions selected based on responses received from the care giver computing device to at least one previously sent question, (iii) send an expert-language representation of the responses received from the care giver computing device to the care provider computing device, (iv) receive a diagnosis and a management protocol from the care provider computing device, and (v) send the management protocol to the care giver computing device.

45. The system of claim 44, wherein the one or more data storage devices store a medical protocol associated with the diagnosis,
the core computing system is further configured to send the medical protocol to the care provider computing device after receiving the diagnosis, receive an acceptance of the medical protocol from the care provider computing device, and
the management protocol comprises at least a portion of the accepted medical protocol.

46. The system of claim 45, wherein the core computing system is further configured to receive modifications to the medical protocol from the care provider, and the management protocol comprises at least a portion of the modified medical protocol.

47. The system of claim 44, wherein the one or more data storage devices store a health profile associated with the care recipient experiencing the medical issue, the health profile comprising health information related to the care recipient, and the core computing system is further configured to select the selected ones of the plurality of data collection questions based at least in part on the health profile associated with the care recipient.

48. The system of claim 44, wherein the core computing system comprises a plurality of computing devices.
